SEQUENCE LISTING (27 sequences)

SEQ ID NO: 1 - protein, 250 aa, Salmonella enterica
MKSRYLVFFLPLIVAKYTSAETVQPFHSPEESVNSQFYLPPPPGNDDPAFRYDKEAYFKGYAIKSSPRWKQAAEDADVSVENIARIFSPVVGAKINPKDTPETWNMLQNLLTMGGYYATASAKKYYMRTRPFVLFNHSTCRPEDENTLRKDGSYPSGHTAYGTLLALVLSEARPERAQELARRGWEFGQSRVICGAHWQSDVDAGRYVGAVEFARLQTIPAFQKSLAKVREELNDKNNLLSKEDHPELNY

SEQ ID NO: 2 - protein, 250 aa, Salmonella enterica; identical to SEQ ID NO: 1

SEQ ID NO: 3 - protein, 250 aa, Salmonella enterica
MKSRYLVFFLPLIVAKYTSAETVQPFHSPEESVNSQFYLPPPPGNDDPAFRYDKEAYFKGYAIKGSPRWKQAAEDADVSVENIARIFSPVVGAKINPKDTPETWNMLQNLLTMGGYYATASAKKYYMRTRPFVLFNHSTCRPEDENTLRKDGSYPSGHTAYGTLLALVLSEARPERAQELARRGWEFGQSRVICGAHWQSDVDAGRYVGAVEFARLQTIPAFQKSLAKVREELNDKNNLLSKEDHPELNY

SEQ ID NO: 4 - protein, 250 aa, Salmonella enterica
MKSRYLVFFLPLIVAKYTSAETVQPFHSPEESVNSQFYLPPPPGNDDPAFRYDKEAYFKGYAIKGSPRWKQAAEDADVSVENIARIFSPVVGAKINPKDTPETWNMLQNLLTMGGYYAIASAKKYYMRTRPFVLFNHSTCRPEDENTLRKDGSYPSGHTAYGTLLALVLSEARPERAQELARRGWEFGQSRVICGAHWQSDVDAGRYVGAVEFARRQTIPAFQKSLAKVREELNDKNNLLSKEDHPELNY

SEQ ID NO: 5 - protein, 250 aa, Salmonella enterica
MKSRYLVFFLPLIVAKYTSAETVQPFHSPEESVNSQFYLPPPPGNDDPAYRYDKEAYFKGYAIKGSPRWKQAAEDADVSVENIARIFSPVVGAKINPKDTPETWNMLKNLLTMGGYYATASAKKYYMRTRPFVLFNHSTCRPEDENTLRKNGSYPSGHTAYGTLLALVLSEARPERAQELARRGWEFGQSRVICGAHWQSDVDAGRYVGAVEFARLQTIPAFQKSLAKVREELNDKNNLLSKEDHPKLNY

SEQ ID NO: 6 - protein, 250 aa, Salmonella enterica
MKSRYLLFFLPLIVAKYTSAETVQPFHSPEESVNSQFYLPPPPGNDDPAFRYDKEAYFKGYAIKGSPRWKQAAEDADVSVENIARIFSPVVGAKINPKDTPETWNMLQNLLTMGGYYATASAKKYYMRTRPFVLFNHSTCRPEDENTLRKDGSYPSGHTAYGTLLALVLSEARPERAQELARRGWEFGQSRVICGAHWQSDVDAGRYVGAVEFARLQTIPAFQKSLAKVREELNDKNNLLSKEDHPELNY

SEQ ID NO: 7 - protein, 250 aa, Salmonella enterica
MKSRYLLFFLPLIVAKYTSAETVQPFHSPEESVNSQFYLPPPPGNDDPAFRYDKEAYFKGYAIKGSPRWKQAAEDADVSVENIARIFSPVVGAKINPKDTPETWNMLQNLLKMGGYYATASAKKYYMRTRPFVLFNHSTCRPEDENTLRKDGSYPSGHTAYGTLLALVLSEARPERAQELARRGWEFGQSRVICGAHWQSDVDAGRYVGAVEFARLQTIPAFQKSLAKVREELNDKNNLLSKEDHPELNY

SEQ ID NO: 8 - protein, 250 aa, Salmonella enterica
MKSRYLLFFLPLIVAKYTSAATVQPFHSPEESVNSQFYLPPPPGNDDPAFLYDKEAYFKGYAIKGSPRWKQAAEDADVSVENIARIFSPVVGAKINPKDTPETWNMLKNLLTMGGYYATASAKKYYMRTRPFVLFNHSTCRPEDENTLRKDGSYPSGHTAYGTLLALVLSEARPERAQELARRGWEFGQSRVICGAHWQSDVDAGRYVGAVEFARLQTIPAFQKSLAKVREELNDKNNLLSKEDHPKLNY

SEQ ID NO: 9 - protein, 250 aa, Salmonella enterica
MKSRYLVFFLPLIVAKYTSAETVQPFHSPEESVNSQFYLPPPPGNDDPAFRYDKEAYFKGYAIKGSPRWKQAAEDADVSVENIARIFSPVVGAKINPKDTPETWNMLKNLLTMGGYYATASAKKYYMRTRPFVLFNHSTCRPEDENTLRKDGSYPSGHTAYGTLLALVLSQARPERAQELARRGWEFGQSRVICGAHWQSDVDAGRYVGAVEFARLQTIPAFQKSLAKVREELNDKNNLLSKEERPELNY

SEQ ID NO: 10 - protein, 250 aa, Salmonella enterica
MKSRYLVFFLPLIVAKYTSAETVQPFHSPEESVNSQFYLPPPPGNDDPAFRYDKEAYFKGYAIKGSPRWKQAAEDADISVENIARIFSPVVGAKINPKDTPETWNMLQNLLKMGGYYATASAKKYYMRTRPFVLFNHSTCRPEDENTLRKNGSYPSGHTAYGTLLALVLSEARPERAQELARRGWEFGQSRVICGAHWQSDVDAGRYVGAVEFARLQTIPAFQKSLAKVREELNDKNNLLSKEDHPELNY

SEQ ID NO: 11 - protein, 250 aa, Salmonella enterica
MKSRYLVFFLPLIVAKYTSAETVQPFHSPEESVNSQFYLPPPPGNDDPAFRYDKEAYFKGYAIKGSPRWKQAAEDADVSVENIARIFSPVVGAKINPKDTPETWNMLKNLLTMGGYYATASAKKYYMRTRPFVLFNHTTCRPEDENTLRKNGSYPSGHTAYGTLLALVLSEARPERAQELARRGWEFGQSRVICGAHWQSDVDAGRYVGAVEFARLQTIPAFQKSLAKVREELNDKNNLLSKEDHPKLNY

SEQ ID NO: 12 - protein, 250 aa, Salmonella enterica
MKSRYLVFFLPLIVAKYTSAETVQPFHSPEESVNSQFYLPPPPGNDDPAFRYDKEAYFKGYAIKGSPRWKQAAEDADVSVENIARIFSPVVGAKINPKDTPETWNMLKNLLTMGGYYATASAKKYYMRTRPFVLFNHSTCRPEDENTLRKDGSYPSGHTAYGTLLALVLSQARPERAQELARRGWEFGQSRVICGAHWQSDVDAGRYVGAVEFARLQTIPAFQKSLAKVREELNDKNNLLSKADHPELNY

SEQ ID NO: 13 - protein, 250 aa, Salmonella enterica
MKSRYLVFFLPLIVAKYTSAATVQPFHSPEESVNSQFYLPPPPGNDDPAFRYDKEAYFKGYAIKGSPRWKQAAEDADISVENIARIFSPVVGAKINPKDTPETWNMLQNLLKMGGYYATASAKKYYMRTRPFVLFNHSTCRPEDENTLRKDGSYPSGHTAYGTLLALVLSEARPERAQELARRGWEFGQSRVICGAHWQSDVDAGRYVGAVEFARLQTIPAFQKSLAKVREELNDKNNLLSKEDHPELNY

SEQ ID NO: 14 - protein, 250 aa, Salmonella enterica
MKSRYLVFFLPLIVAKYTSAETVQPFHSPEESVNSQFYLPPPPGNDDPAFRYDKEAYFKGYAIKGSPRWKQAAEDADISVENIARIFSPVVGAKINPKDTPETWNMLQNLLKMGGYYATASAKKYYMRTRPFVLFNHSTCRPEDENTLRKDGSYPSGHTAYGTLLALVLSEARPERAQELARRGWEFGQSRVICGAHWQSDVDAGRYVGAVEFARLQTIPAFQKSLAKVREELNDKNNLLSKEDHPKLNY

SEQ ID NO: 15 - protein, 250 aa, Salmonella enterica
MKSRYLLFFLPLIVAKYTSAATVQPFHSPEESVNSQFYLPPPPGNDDPAFLYDKEAYFKGYAIKGSPRWKQAAEDADVSVENIARIFSPVVGAKINPKDTPETWNMLKNLLTMGGYYATASAKKYYMRTRPFVLFNHSTCRPEDENTLRKDGSYPSGHTAYGTLLALVLSEARPERAQELARRGWEFGQSRVICGAHWQSDVDAGRYVGAVEFARLQTIPAFQKSLAKVREELNDKNNLLSKEDHPELNY

SEQ ID NO: 16 - protein, 250 aa, Salmonella enterica
MKSRYLLFFLPLIVAKYTSAATVQLFHSPEESVNSQFYLPPPPGNDDPAFRYDKEAYFKGYAIKGSPRWKQAAEDADVSVENIARIFSPVVGAKINPKDTPETWNMLQNLLKMGGYYATASAKKYYMRTRPFVLFNHSTCRPEDENTLRKDGSYPSGHTAYGTLLALVLSQARPERAQELARRGWEFGQSRVICGAHWQSDVDAGRYVGAVEFARLQTIPAFQKSLAKVREELNDKNNLLSKEDHPKLNY

SEQ ID NO: 17 - protein, 250 aa, Salmonella enterica
MKSRYLLFFLPLIVAKYTSAETVQPFHSPEESVNSQFYLPPPPGNDDPAFRYDKEAYFKGYAIKGSPRWKQAAEDADVSVENIARIFSPVVGAKINPKDTPETWNMLQNLLKMGGYYATASAKKYYMRTRPFVLFNHSTCRPEDEDTLRKDGSYPSGHTAYGTLLALVLSQARPERAQELARRGWEFGQSRVICGAHWQSDVDAGRYVGAVEFARLQTIPAFQKSLAKVREELNDKNNLLSKEDHPKLNY

SEQ ID NO: 18 - protein, 250 aa, Salmonella enterica
MKSRYLLFFLPLIVAKYTLAATVQLFHSPEESVNSQFYLPPPPGNDDPAFRYDKEAYFKGYAIKGSPRWKQAAEDADVSVENIARIFSPVVGAKINPKDTPETWNMLQNLLKMGGYYATASAKKYYMRTRPFVLFNHSTCRPEDENTLRKDGSYPSGHTAYGTLLALVLSQARPERAQELARRGWEFGQSRVICGAHWQSDVDAGRYVGAVEFARLQTIPAFQKSLAKVREELNDKNNLLSKEDHPKLNY

SEQ ID NO: 19 - protein, 250 aa, Salmonella enterica
MKSRYLLFFLPLIVAKYTSAATVQLFHSPEESVNSQFYLPPPPGNDDPAFRYDKEAYFKGYAIKGSPQWKQAAEDADVSVENIARIFSPVVGAKINPKDTPETWNMLQNLLKMGSYYATASAKKYYMRTRPFVLFNHSTCRPEDENTLRKDGSYPSGHTAYGTLLALVLSQARPERAQELARRGWEFGQSRVICGAHWQSDVDAGRYVGAVEFARLQTIPAFQKSLAKVREELNDKNNLLSKEERPELNY

SEQ ID NO: 20 - protein, 250 aa, Salmonella enterica
MKSRYLLFFLPLIVAKYTSAATMQPFHSPEESVNSQFYLPPPPGNDDPAFRYDKEAYFKGYAIKGSPRWKQAAEDADISVENIARIFSPVVGAKINPKDTPETWNMLQNLLKMGGYYATASAKKYYMRTRPFVLFNHSTCRPEDENTLRKDGSYPSGHTAYSTLLALVLSQARPERAQELARRGWEFGQSRVICGAHWQSDVDAGRYVGAVEFARLQTIPAFQKSLAKVREELNDKNNLLSKEERPELNY

SEQ ID NO: 21 - protein, 249 aa, Escherichia blattae
MKKRVLAVCFAALFSSQALALVATGNDTTTKPDLYYLKNSEAINSLALLPPPPAVGSIAFLNDQAMYEQGRLLRNTERGKLAAEDANLSSGGVANAFSGAFGSPITEKDAPALHKLLTNMIEDAGDLATRSAKDHYMRIRPFAFYGVSTCNTTEQDKLSKNGSYPSGHTSIGWATALVLAEINPQRQNEILKRGYELGQSRVICGYHWQSDVDAARVVGSAVVATLHTNPAFQQQLQKAKAEFAQHQKK

SEQ ID NO: 22 - protein, 249 aa, Shigella dysenteriae
MKRQLFTLSIVGVFSLNTFASFPPGNDVTTKPDLYYLTNDNAIDSLALLPPPPQIGSIAFLNDQAMYEKGLLLRNTERGKLAAEDANLSSGGVANVFSAAFGSPITAKDSPELHKLLTNMIEDAGDLATRSAKEYYMRIRPFAFYGVSTCNTKEQDTLSRNGSYPSGHTSIGWATALVLSEINPARQDTILKRGYELGDSRVICGYHWQSDVDAARIVGSAIVATLHSNPVFQAQLQKAKDEFANNQKK

SEQ ID NO: 23 - DNA, 2750 bp, Salmonella enterica
cgaaaccctgaacctgcgtatcaaacccgccgagcgcgatcttattgatcgagcggcgaaagccagagggaaaaaccgcactgactttgtgctggaagctgcccgtgctgccgccgaggaagcgctgatcgaacaacgcatcattatggccgatccagaggcttatcaggagtttctcgttcgtttggatcaaacaccttcacctaatgccgcactgcgtaaaaccatgcagacccctgcgccgtgggagcaggaaaaatgatctccacccctgagccgcttcatgccggacatattcttactccgttttgctgcggtgtggattctatagataactggctgaaacagcgggcgatgaaaaatcagaccactggcgcgtcccgtacctttgtgtgttgtggcagcgattcgaacgtactggcctattactcgctggcgtccagcgcggtcacgacgaatacctcccccggtcgctttcgtcgcaatatgcctgacccgattccggttgtggtattggggcgtctggcggtggataaatcgcttcatgggcagggcgtcgctcgggcgctggtacgcgacgcagggctgcgggttattcaggtggcggagactatcggcattcgtgggatgctggttcacgccctgtcggatgaagcgcgggaattttatcagcgggtggggtttgtaccgtcgccgatggatccgatgatgttgatggtgacgttgggggatttggtggagagtgtttaaactccagaacgcatcccttagcttctgatggccggaattcgctgtggatagtcagtgagccagaccggttttaccgcttcagccggacaacgacgtcatgacggtctggacggactgcaaacgggggaagggggcgttgaattcatgcgaatcggggaaaccaaaggcattctccacgcccggccgctcctacgcagttgcacttcctttcatttgctgtggccagtttgcgggaagactttcaccttcagtaattaagttcggggtgatcttctttactcaataaattatttttgtcgttcagctcctcacggacttttgccagtgacttctgaaatgccgggattgtttgcagtcttgcaaactctactgctcccacataacgaccagcatcaacatcgctttgccagtgagcaccgcatatcactctgctttgcccgaactcccatcctcgtctggcgagctcctgcgcgcgttccggtctggcctcggataatactaatgccagaagtgtaccataagcagtatgcccggaagggtaagagccatcttttcgcaaagtattctcatcttcaggacggcaggtggaatgattaaataagacaaagggacgagtgcgcatataatatttttttgccgaagcagtagcgtagtagccgcccattgtcagaagattctgtaacatattccaggtttctggcgtatctttgggattaattttagcacccactactggcgagaatattctggctatattttccacgcttacatctgcatcctcagcagcttgtttccatcgcggggaactctttatcgcatagcccttaaaataagcctccttatcatagcggaaagccggatcatcattacctggcggtggtggtaagtagaactgactgttcactgattcttcaggagaatgaaagggttgcactgtttctgctgatgtatatttagctacgatcagtggtagaaaaaatactaaataacgacttttcataaagactcactccggatcaggcaataataaacagatgtaatcctataatgatttattgcaatggttattcaacatacaggtaaaaaagcaggacgctgcactggtattattgtctgtccataccggactgactgtagcggacaacagaacaataccggtacttccggctaacagcattttgtttaaattttctccccggttgttttttatctgcgtgcctgactgacacgcattaggttaaaatctttccgtaataacaaactatgaataagtattttaaagtatgtgctctgttaccccggtaactaccatcagtcatgatgtgctgagtattaatttatgattatgaacaagagtcggaaacagtatttaagaataaagaagccgacaaagaaaaaatgccaaaactggcgaagggattattgttttcagtgatgaaaaagttcatggcggagatattggactcacttgtcggggttagcgaaccgtcagatgaagtgataggcgttattactggtaataatctccctgtagctaagtaagcttttgaccctagcccaaaggtgagcggctgatcgacgaagcgctgggcgttgcgccagagaaaatctggccttctcgttatagctagtcagaccagagatgggggctacggatttccattcccagaaaagcaaaaacccagccataggctggattttctgaataagtggtgcccggactcggaatcgaaccaaggacacggggattttcaatcccctgctctaccgactgagctatccgggcaacggggcgcattaaaccgtaaaggggcgcggtcgtcaacggctttacacgaaaaaagtgataaaagcgcgctgactgcctgcttttcaggcaaaatgcataaaatcagtacccgtcaggtgagcgcgccaccctgacggcactgtgcaaaacgcagaatatcttccgccaaccgctgagcggtatctacatcagcctgactgcggttgaccag

SEQ ID NO: 24 - DNA, 2750 bp, Salmonella enterica
cgaaaccctgaacctgcgtatcaaacccgccgagcgcgatcttattgatcgagcggcgaaagccagagggaaaaaccgcactgactttgtgctggaagccgcccgtgctgccgccgaggaagcgctgatcgaacaacgcatcattatggccgatccagaggcctatcaggaatttctggctcgtttggatcaaacaccttcacctaatgccgcactgcgtaaaaccatgcagacccctgcgccgtgggagcaggaaaaatgatctcctcccctgagccgcttcatgccggacatattcttactccgttttgctgcggtgtggattctatggataactggctgaaacagcgggcgatgaaaaatcagaccactggtgcgtcccgtacctttgtgtgttgtggcagcgattcgagcgtactggcctattactcgctggcgtccagcgcggtcacgacgaataccgcccccggtcgctttcgtcgcaatatgcctgacccgattccgattgtggtattggggcgtctggcggtggataaatcgcttcatgggcagggcgttggtcgggcgctggtacgcgacgcagggctgcgggttattcaagtggcggagactatcggcattcgtgggatgctggttcacgccctgtcggatgaagcgcgggaattttatcagcgggtggggtttgtaccgtcgccgatggatccgatgatgttgatggtgacgttgggggatttggtggagagtgtttaaactccagaacgcatcccttagcttctgatggccggaattcgctgtggatagtcagtgagccagaccggttttaccgcttcagccggacaacgacgtcatgacggtctggacggactgcaaacgggggaagggggcgttgaattcatgcgaatcggggaaaccaaaggcattctccacgcccggccgctcctacgcagttgcacttcctttcatttgctgtggccagtttgcgggaagactttcaccttcagtaattaagttcggggtgatcttctttactcaataaattatttttgtcgttcagctcctcacggacttttgccagtgacttctgaaatgccgggattgtttgcagtcttgcaaactctactgctcccacataacgaccagcatcaacatcgctttgccagtgagcaccgcatatcactctgctttgcccgaactcccatcctcgtctggcgagctcctgcgcgcgttccggtctggcctcggataatactaatgccagaagtgtaccataagcagtatgcccggaagggtaagagccatcttttcgcaaagtattctcatcttcaggacggcaggtggaatgattaaataagacaaagggacgagtgcgcatataatatttttttgccgaagcagtagcgtagtagccgcccattgtcagaagattctgtaacatattccaggtttctggcgtatctttgggattaattttagcacccactactggcgagaatattctggctatattttccacgcttacatctgcatcctcagcagcttgtttccatcgcggggaaccctttatcgcatagcccttaaaataagcctccttatcatagcggaaagccggatcatcattacctggcggtggtggtaagtagaactgactgttcactgattcttcaggagaatgaaagggttgcactgtttctgctgatgtatatttagctacgatcagtggtagaaaaaatactaaataacgacttttcataaagactcactccggatcaggcaataataaacagatgtaatcctataatgatttattgcaatggttattcaacatacaggtaaaaaagcaggacgctgcactggtattattgtctgtccataccggactgactgtagcggacaacagaacaataccggtacttccggctaacagcattttgtttaaattttctccccggttgttttttatctgcgtgcctgactgacacgcattaggttaaaatctttccgtaataacaaactatgaataagtattttaaagtatgtgctctgttaccccggtaactaccatcagtcatgatgtgctgagtattaatttatgattatgaacaagagtcggaaacagtatttaagaataaagaagccgacaaagaaaaaatgccaaaactggcgaagggattattgttttcagtgatgaaaaagttcatggcggagatattggactcacttgtcggggttagcgaaccgtcagatgaagtgataggcgttattactggtaataatctccctgtagctaagtaagcttttgaccctagcccaaaggtgagcggctgatcgacgaagcgctgggcgttgcgccagagaaaatctggccttctcgttatagctagtcagaccagagatgggggctacggatttccattcccagaaaagcaaaaacccagccataggctgggttttctgaataagtggtgcccggactcggaatcgaaccaaggacacggggattttcaatcccctgctctaccgactgagctatccgggcaacggggcgcattaaaccgtaaaggggcgcggtcgtcaacggctttacacgaaaaaagtgataaaagcgcgctgactgcctgcttttcaggcaaaatgcataaaatcagtacccgtcaggtgagcgcgccaccctgacggcactgtgcaaaacgcagaatatcttccgccaaccgctgagcggtatctacatcagcctgactgcggttgaccag

SEQ ID NO: 25 - DNA, 2750 bp, Salmonella enterica
cgaaaccctgaacctgcgtatcaaacccgccgagcgcgatcttattgatcgagcggcgaaagccagagggaaaaaccgcactgactttgtgctggaagctgcccgtgctgccgccgaggaagcgctgatcgaacaacgcatcattatggccgatccagaggcttatcaggagtttctcgttcgtttggatcaaacaccttcacctaatgccgcactgcgtaaaaccatgcagacccctgcgccgtgggagcaggaaaaatgatctccacccctgagccgcttcatgccggacatattcttactccgttttgctgcggtgtggattctatagataactggctgaaacagcgggcgatgaaaaatcagaccactggcgcgtcccgtacctttgtgtgttgtggcagcgattcgaacgtactggcctattactcgctggcgtccagcgcggtcacgacgaatacctcccccggtcgctttcgtcgcaatatgcctgacccgattccggttgtggtattggggcgtctggcggtggataaatcgcttcatgggcagggcgtcgctcgggcgctggtacgcgacgcagggctgcgggttattcaggtggcggagactatcggcattcgtgggatgctggttcacgccctgtcggatgaagcgcgggaattttatcagcgggtggggtttgtaccgtcgccgatggatccgatgatgttgatggtgacgttgggggatttggtggagagtgtttaaactccagaacgcatcccttagcttctgatggccggaattcgctgtggatagtcagtgagccagaccggttttaccgcttcagccggacaacgacgtcatgacggtctggacggactgcaaacgggggaagggggcgttgaattcatgcgaatcggggaaaccaaaggcattctccacgcccggccgctcctacgcagttgcacttcctttcatttgctgtggccagtttgcgggaagactttcaccttcagtaattaagttcggggtgatcttctttactcaataaattatttttgtcgttcagctcctcacggacttttgccagtgacttctgaaatgccgggattgtttgccgtcttgcaaactctactgctcccacataacgaccagcatcaacatcgctttgccagtgagcaccgcatatcactctgctttgcccgaactcccatcctcgtctggcgagctcctgcgcgcgttccggtctggcctcggataatactaatgccagaagtgtaccataagcagtatgcccggaagggtaagagccatcttttcgcaaagtattctcatcttcaggacggcaggtggaatgattaaataagacaaagggacgagtgcgcatataatatttttttgccgaagcaatagcgtagtagccgcccattgtcagaagattctgtaacatattccaggtttctggcgtatctttgggattaattttagcacccactactggcgagaatattctggctatattttccacgcttacatctgcatcctcagcagcttgtttccatcgcggggaaccctttatcgcatagcccttaaaataagcctccttatcatagcggaaagccggatcatcattacctggcggtggtggtaagtagaactgactgttcactgattcttcaggagaatgaaagggttgcactgtttctgctgatgtatatttagctacgatcagtggtagaaaaaatactaaataacgacttttcataaagactcactccggatcaggcaataataaacagatgtaatcctataatgatttattgcaatggttattcaatatacaggtaaaaaagcaggacgctgcactggtattattgtctgtccataccggactgactgtagcggacaacagaacaataccggtacttccggctaacagcattttgtttaaattttctccccggttgttttttatctgcgtgcctgactgacacgcattaggttaaaatctttccgtaataacaaactatgaataagtattttaaagtatgtgctctgttaccccggtaactaccatcagtcatgatgtgctgagtattaatttatgattatgaacaagagtcggaaacagtatttaagaataaagaagccgacaaagaaaaaatgccaaaactggcgaagggattattgttttcagtgatgaaaaagttcatggcggagatattggactcacttgtcggggttagcgaaccgtcagatgaagtgataggcgttattactggtaataatctccctgtagctaagtaagcttttgaccctagcccaaaggtgagcggctgatcgacgaagcgctgggcgttgcgccagagaaaatctggccttctcgttatagctagtcagaccagagatgggggctacggatttccattcccagaaaagcaaaaacccagccataggctgggttttctgaataagtggtgcccggactcggaatcgaaccaaggacacggggattttcaatcccctgctctaccgactgagctatccgggcaacggggcgcattaaaccgtaaaggggcgcggtcgtcaacggctttacacgaaaaaagtgataaaagcgcgctgactgcctgcttttcaggcaaaatgcataaaatcagtacccgtcaggtgagcgcgccaccctgacggcactgtgcaaaacgcagaatatcttccgccaaccgctgagcggtatctacatcagcctgactgcggttgaccag

SEQ ID NO: 26 - DNA, 2608 bp, Salmonella enterica
cgaaaccctgaacctgcgtatcaaacccgccgagcgcgatcttatcgatcgagcggcgaaagccagagggaaaaaccgcacagactttgtgctggaagctgcccgcgctgccgcagaggaagcgcttatcgaacaacgcatcattatggccgatccagaggcttatcaggagtttctcgttcgtttggatcaaacaccttcacctaatgccgcactgcgtaaaaccatgcagacccctgcgccgtgggagcaggaaaaatgatctccacccctgagccgcttcatgccggacatattcttactccgttttgctgcggtgtggattctatagataactggctggaacagcgggcgatgaaaaatcagaccactggcgcgtcccgtacctttgtgtgttgtggcagcgattcgaacgtactggcctattactcgctggcgtccagcgcggtcacgacgaatacctcccccggtcgctttcgtcgcaatatgcctgacccgattccggttgtggtattggggcgtctggcggtggataaatcgcttcatgggcagggcgtcgctcgggcgctggtacgcgacgcagggctgcgggttattcaggtggcggagactatcggcattcgtgggatgctggttcacgccctgtcggatgaagcgcgggaattttatcagcgggtggggtttgtaccgtcgccgatggatccgatgatgttgatggtgacgttgggggatttggtggagagtgtttaaactccagaacgcatcccttagcttctgatggccggaattcgctgtggatagtcagtgagccagaccggttttaccgcttcagccggacaacgacgtcatgacggtctggacggactgcaaacgggggaagggggcgttgaattcatgcgaatcggggaaaccaaaggcattctccacgcccggccgcccctacgcagttgcacttcctttcatttgctgtggccagtttgcgggaagactttcaccttcagtaattaagtttggggtgatcttctttactcaataaattatttttgtcgttcagctcctcacggacttttgccagtgacttctgaaaagccgggattgtttgcagtcttgcaaactctactgctcccacataacggccagcatcaacatcgctttgccagtgagcaccgcatatcactctgctttgcccgaactcccatccgcgtctggcgagctcctgcgcgcgttccggtctggcctcggataatactaatgccagaagtgtaccataagcagtatgcccggaagggtaagagccattttttcgcaaagtattctcatcttcaggacggcaggtggaatgattaaataagacaaaggggcgggtacgcatataatatttttttgccgaagcagtagcgtagtagccgcccattgtcagaagattctttaacatattccaggtttctggcgtatctttggggttaattttagcacccactactggcgagaatattctggctatattttccacgcttacatctgcatcctcagcagcttgtttccatcgcggggaaccctttatcgcatagcccttaaaataagcctccttatcatagcggtaagccggatcatcattacctggcggtggtggtaagtagaactgactgttcactgattcttcaggagaatgaaagggttgcactgtttctgctgatgtatatttagctacgatcagtggtagaaaaaatactaaataacgacttttcataaagactcactccggatcaggcaataataaacagatgtaatcctataatgatttattgcaatggttattcaacatacaggtaaaaaagcaggacgctgccctggcattatcgtctgtccataccggactgactgcagcggacaacagaacaataccggtacttccggctaacagcattttgtttaacttttgtccccgattattttttatctacgtgcctgactgacacgcattaggttaaaatctttccgtaataacacagtatgaataattattttaaagtatgtgctctgttaccccgataactaccatcagtcatgatgtgctgagtattaatttatgattatgaacaagagtcggaaacagtatttaagaataaagaaaccgaaaaagaaaaaatgctaaaattggcgaagggattattgttatcagtgatgaaaaagttcatggcggagatattggattcacttgtcggggttagcgaaccgtcagatggagtgatacgcgttattactggtaataatctccctgtagctaagtaagctttcctgacaggcgtgataaaagttccgcgcgataaaaaggcttgtacctggcattataagactcgcgtcctgcagaaacagggcagggaagggctcggcggttttaatcaggtatatctgttaaatcacatccttcaactggagcagcctttgttttaaccttcctctcatcgggccggaatgcttcgcctgcgttgatgtagcagaaaccggagaaatcacgccatccgatttcttgcaaatatcaggcgcaaaaaaacagacttgcgctaacaagtctgttctttaagagcctttcttttaacaacaagta

SEQ ID NO: 27 - DNA, 2750 bp, Salmonella enterica
attcgactgcgtttgtagtgtagatggtagatggtcttgcggatatccggcggataactgaagagcgtattgaggttttcccagtgcgcacgccagcttttgctgatttgcggatatttcgggaagacgctgtttatcgcatccgggaagcccttcagaccgtccacacaggcaatcaggatgtcctgaaggccccgctttttcagctccgtcagcacgcttagccagaactttgtgccttcatttccggacagccacatgccggcaacgctttctgaccttcggtattgatccccagcgcagggaaaactgctttgtttatcacactgccattctgatgaactttgacaacatacagtccatataaacaaagggatacagcgtatccatttgtcggttttgccactcaatgacctgctcttttacggcgtcggtgactttatatatcagcgtgggcgacacatcggagtcgtacatttctttgaaggtggcaacgatttcgcggatagtcatgcctttggcgcagagggataaaatctggctgtccatctgcgtaatgcgcgtctggtgcttcttaatcagctgtggttcgaaggtattttcacggtcacgcggcgtgttcagttcgatctcgctgttattgcataacaccgtttttgacgagtagccattgcgggtgtttgagcctgttttgggcgcatttttctcatacccggggtggtcagccagctccgcattgagcgctgtttcgacggtaagcttcgtcaacatgcgggaaaaagcatggagatcggtttcggttttaaggccttcagccacttcagccgcaagggctttaagtttcttttcgtccataattgcctgtctctgttgttggagtgaacatatcaaaaacaggcacctacacaattttaattacagtctaacgcccggtcacccctacgcagttgcacttcctttcatttgctgtggccagtttgcgggaagactttcaccttcagtaattaagtttgggatgatcttctttactcaataaattatttttgtcgttcagctcctcacggacttttgccagtgacttctgaaaagccgggattgtttgcagtcttgcaaactctactgctcccacataacggccagcatcaacatcgctttgccagtgagcaccgcatatcactctgctttgcccgaactcccatcctcgtctggcgagctcctgcgcgcgttccggtctggcctcggataatactaatgccagaagtgtaccataagcagtatgcccggaagggtaagagccatcttttcgcaaagtattctcatcttcaggacggcaggtagaatgattaaataagacaaagggacgagtacgcatataatatttttttgccgaagcagtcgcgtagtagccgcccattgtcagaagattctttaacatattccaggtttctggcgtatctttggggttaattttagcacccactactggcgagaatattctggctatattttccacgcttacatctgcatcctcagcagcttgtttccatcgtggggaaccctttatcgcatagcccttaaaataagcctccttatcatagaggaaagccggatcatcattacctggcggtggtggtaagtagaactgactgttcactgattcttcaggagaatgaaagggttgcactgttgctgctgatgtatatttagctacgatcagtggtagaaaaaatagtaaataacgacttttcataaagactcgctccggatcagacaataataaacagatgtaatcctataatgatttattgcaatggttattcaacatacaggtaaaaaagcaggacgctgcactggtattattgtctgtccataccggactgactgcagcggacaacagaacaataccggtacttccggctaacagcattttgtttaatttttctccccggttattttttatctgcgtgcctgactgacacgcattaggttaaaatgtttccgtaatagcaaactatgaataagtattttaaagtatgtgctctgttaccccggtaactaccatcagtcatgatgtgctgagtattaatttatgattatgaacaagagtcggaaacagtatttaagaataaagaagccgacaaagaaaaaatgccaaaactggcgaagggattattgttttcagtgatgaaaaagttcatggtggagatattggactcacttgtcggggttagcgaaccgtcagatgaagtgataggcgttattactggtaataatctccctgtagctaagtaagcttttgaccctggcccaaaggggaaaagctgatcgccgaagcgctgggcgttgcgccagagaaaatctggccttctcgttatagctagtcagaccagagatgggggctacggatttccattcccagaaaagcaaaaacccagccataggctgggttttctgaataagtggtgcccggactcggaatcgaaccaaggacacggggattttcaatcccctgctctaccgactgagctatccgggcaacggggcgcattaaaccgtaaaggggcgcggtcgtcaacggctttacacgaaaaaagtgataaaagcgcgctgactgcctgcttttcaggcaaaatgcataaaatcagtacccgtcaggtgagcgcgccaccctgacggcactgtgcaaaacgcagaatatcttccgccaaccgctgagcggtatctacatcagcctgactgcggttgaccag

(19) United States
(12) Patent Application Publication
(10) Pub. No.: US 2012/0114698 A1
(43) Pub. Date: May 10, 2012

(54) SALMONELLA MARKER VACCINE

(76) Inventor: Heiko Apfel, Pulheim (DE)

(21) Appl. No.: 13/129,844

(22) PCT Filed: Nov. 20, 2009
(86) PCT No.: PCT/EP2009/065571

§ 371 Date: Aug. 15, 2011

(30) Foreign Application Priority Data
Nov. 20, 2008 (EP) 08020242.7

(51) Int. Cl. A61K 39/112; G01N 33/569; C12N 1/20; A61P 37/04; C07K 14/255; C12N 15/31

(52) U.S. Cl. 424/258.1; 530/350; 536/23.7; 435/252.8; 435/7.35

(57) ABSTRACT

The present invention relates to a Salmonella strain suitable as a marker vaccine having an inactivated gene.

The present invention relates to a Salmonella strain having an inactivated phoN gene. In particular, the invention relates to a method for the preparation of ΔphoN Salmonella enterica live-attenuated vaccine strains. Further, the invention relates to a serological test system which uses the recombinant Salmonella protein PhoN for discrimination of vaccinated animals from Salmonella infected ones. Furthermore a bacteriological test system is disclosed for DIVA.

The invention further concerns the Salmonella enterica ssp. enterica serovar Enteritidis strain CLAB SE404 (DSM 21972) and its use as a live-attenuated ΔphoN Salmonella marker vaccine strain for the treatment of poultry to prevent Salmonella Enteritidis infection and/or for the preparation of polyvalent recombinant Salmonella carrier vaccines which protect livestock against additional pathogens. CLAB SE404 enables the Differentiation of infected from Vaccinated Animals (DIVA) by serological or/and bacteriological means.

Nontyphoidal Salmonellosis is a widespread disease in animals and humans. In men it is the most common food-borne disease which mainly originates from products of poultry and pigs. In the European Union a surveillance system will be established which should reduce the intake of pathogenic Salmonella ssp. into the food chain (regulation EC 2160/2003). Vaccination may reduce the prevalence of pathogenic Salmonella ssp. in livestock and thus minimize the transmission rate. Anti-Salmonella vaccines are commercially available e.g. for laying hens and pigs. The vaccines are either bacterins or live-attenuated Salmonella vaccines. In laying hens, these vaccines are used to reduce intra-ovarian transmission and fecal shedding of the pathogen.

The efficacy of live-attenuated Salmonella vaccines has been found superior to bacterins. The preferred use of live-attenuated Salmonella vaccines for livestock, demands a method for the Differentiation of infected from Vaccinated Animals (DIVA) which complies with the regulations of EC 2160/2003.

The majority of the authorized live-attenuated Salmonella vaccines are prepared from defined wildtype isolates of Salmonella enterica ssp. enterica serovars by chemical mutagenesis. Presently, live-attenuated Salmonella vaccines are distinguished from wildtype Salmonella ssp. by bacteriological means. These processes are long lasting and laborious. A serological discrimination of vaccinated and infected animals would be highly preferable. The presently available live-attenuated Salmonella vaccines do not enable a serological discrimination according to DIVA strategy.

DIVA live vaccines have been successfully used in livestock for the prevention of viral infections.

A marker vaccine can be prepared either as positive or negative marker. A positive marker vaccine contains an additional antigen which induces specific antibodies in vaccinated animals but not in infected ones. Positive marker vaccines are clearly disadvantageous because vaccinated animals with a subsequent infection cannot be discriminated from solely vaccinated ones.

A negative marker vaccine is prepared by removal of an antigen which provokes specific antibodies in infected animals. Thus, a negative marker vaccine provokes a humoral immune response in vaccinated animals that is different from that of infected animals. Ideally, this difference can be detected by serological means. In contrast to a positive marker vaccine, a negative marker vaccine enables the identification of vaccinated animals which become subsequently infected.

For the selection of a negative marker antigen several aspects must be considered: The antigen must be immunogenic but not be involved in the immune processes which protect animals against the pathogen. Thus the removal or inactivation of the gene which encodes the marker antigen must maintain immunogenicity of the vaccine strain. Finally, the marker antigen should be specific for the pathogen in order to avoid false-positive serological results which are induced by other organisms that may appear in livestock and provoke a humoral immune response.

Due to the high complexity of live-attenuated bacterial vaccines in contrast to viral vaccines much more effort is needed for the development of a live-attenuated bacterial marker vaccine. Live-attenuated bacterial marker vaccines are still at an experimental stage. The first candidates of a negative Salmonella marker vaccine were established from live-attenuated Salmonella vaccine strains by deleting the genes of highly immunogenic surface antigens. These live-attenuated Salmonella DIVA vaccines were able to differentiate infected from vaccinated animals by serological means. However, these prototypes were less protective than the parental vaccine strain.

The gene phoN encodes a non-specific acid phosphatase, which has been found widely preserved in all Salmonella ssp. The gene phoN is expressed under the control of the PhoPQ regulon, which is a general regulator for virulence genes in Salmonella. It is not known whether phoN is essential for the survival of Salmonella in animals.

The linkage of phoN to the PhoPQ regulon may indicate that phoN becomes activated whenever Salmonella occupies the host's tissue. Thus the question remains whether the elimination or inactivation of the gene phoN in a live-attenuated Salmonella vaccine strain may negatively affect its potential to induce protective immunity in vaccinated animals. It is known that the depletion of a single gene in a live-attenuated Salmonella vaccine strain may further reduce its residual virulence which may diminish the stimulation of the host's immune response and as a consequence may abrogate protective immunity.

It is the problem of the present invention to provide a Salmonella strain suitable as a negative marker vaccine which at least partially overcomes the above described disadvantages of state of the art vaccines.

In the present invention, the gene phoN has been selected as a target for the preparation of a Salmonella DIVA vaccine. Surprisingly, the phoN gene provides a combination of properties that make a phoN-deficient Salmonella strain especially suitable as a live vaccine.

The first surprising property is the different structure of the PhoN protein in all non-Salmonella bacteria compared with Salmonella. On the other hand, the PhoN amino acid sequence has a degree of at least 95% identity (calculated over the total length of the amino acid sequence) in different Salmonella species.

The second surprising property is the fact that PhoN is immunogenic. Analyses of sera from chicken and mice infected with Salmonella ssp. clearly revealed the appearance of antibodies which specifically bind to purified PhoN protein. This result provides the first evidence that PhoN is immunogenic and can be used as serological marker antigen for detecting Salmonella-infected birds and mammals. If the expression of the gene phoN is abolished in any Salmonella vaccine strain, this might allow the discrimination of infected animals from vaccinated ones by serological means. Thus, animals vaccinated by a Salmonella live vaccine strain with depleted PhoN antigen, will not produce PhoN-specific antibodies, whilst animals which become infected with wildtype Salmonella would do so.

The third surprising property is the fact that the removal of the gene phoN of a live-attenuated Salmonella vaccine strain may further reduce the virulence of the vaccine strain but does not affect its immunogenicity and its efficacy to protect vaccinated chicken from Salmonella infection of homologous serovar. A first subject of the present invention is a Salmonella strain having an inactivated phoN gene. This in indicated herein by ΔphoN. A ΔphoN Salmonella strain has essentially no phoN activity. The person skilled in molecular biology knows methods of gene inactivation in bacteria such as Salmonella.

Inactivation of phoN includes deletion or/and modification of the phoN gene. In particular, the Salmonella strain of the present invention produces essentially no PhoN protein or a fragment thereof which is capable to induce an immune response against the PhoN protein or a fragment thereof, i.e. the Salmonella strain of the present invention is not immunogenic with respect to the PhoN protein or a fragment thereof. The phoN gene may be deleted completely or at least partially. Partial deletion of the phoN gene may be a deletion of at least 50%, at least 60%, at least 70%, at least 80%, at least 90%, or at least 95%, or at least 98% of the full-length sequence of the phoN gene. If the phoN gene sequence is deleted partially, the remaining sequences are preferably not be able to express a PhoN protein or a fragment thereof, in particular an immunogenic fragment as described herein.

Modification of the phoN gene sequence includes sequence replacement. The phoN gene may be completely replaced by another sequence. Deleted sequences of the phoN gene may be replaced completely or partially by other sequences.

The coding sequence (open reading frame) of the phoN gene may be deleted completely or at least partially. Partial deletion of the phoN coding sequence may be a deletion of at least 50%, at least 60%, at least 70%, at least 80%, at least 90%, or at least 95%, or at least 98% of the sequence of the full-length phoN coding sequence. If the phoN coding sequence is deleted partially, the remaining sequences are preferably not be able to express a PhoN protein or a fragment thereof, in particular an immunogenic fragment as described herein.

Modification of the phoN coding sequence includes sequence replacement. The phoN coding sequence may be completely replaced by another sequence. Deleted sequences of the phoN coding sequence may be replaced completely or partially by other sequences.

Typical examples of sequence of the phoN polypeptide in Salmonella are described in FIG. 1 (SEQ ID NO:1 to 20). Typical examples of the Salmonella phoN gene locus are described in FIG. 2 including the phoN open reading frames (ORF) and regulatory sequences upstream or/and downstream of the phoN ORF (SEQ ID NO:23 to 27).

Fragments of the phoN polypeptide of the present invention may have a length of a least 10, at least 20, or at least 30 amino acid residues. They may have a length of at the maximum 200, at the maximum 150, or at the maximum 100 amino acid residues. Fragments include immunogenic fragments (also termed herein as immunogenic portions).

Inactivation of the phoN gene is preferably irreversible inactivation, such as by complete or at least partial deletion of the phoN coding region, or by replacement of the phoN coding region, as described herein. In particular, irreversible inactivation prevents the Salmonella strain of the present invention from reverting to the wildtype phoN genotype.

As indicated above, the Salmonella strain of the present invention produces essentially no PhoN protein which is capable to induce an immune response against the PhoN protein, i.e. the Salmonella strain of the present invention is not immunogenic with respect to the PhoN protein or a fragment thereof. Immunogenicity with respect to the PhoN protein or a fragment thereof may be an immunogenicity in any species to be treated with the Salmonella strain of the present invention. In particular, immunogenicity with respect to the PhoN protein or a fragment thereof is immunogenicity in mammals, e.g. pigs, or birds, e.g. poultry such as chickens.

The person skilled in the art is able to identify the PhoN polypeptide and the phoN gene in Salmonella or non-Salmonella strains. In the present invention, the Salmonella PhoN protein may be a protein comprising a sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:18, SEQ ID NO:19, and SEQ ID NO:20, or a polypeptide comprising a sequence which is at least 70%, at least 80%, at least 90%, at least 95%, at least 98%, or at least 99% identical to the selected sequence, wherein identity is calculated relative to the length of the selected sequence. A non-Salmonella PhoN protein may be a PhoN protein obtained from Escherichia or Shigella and may have a sequence selected from SEQ ID NO:21 and SEQ ID NO:22, or a polypeptide comprising a sequence which is at least 70%, at least 80%, at least 90%, at least 95%, at least 98%, or at least 99% identical to a sequence selected from SEQ ID NO:21 and SEQ ID NO:22, wherein identity is calculated relative to the length of the selected sequence.

The Salmonella strain which is subject to phoN inactivation as described herein in order to obtain a strain according to the present invention may be selected from Salmonella enterica ssp. It is preferred that the Salmonella strain is selected from serovars of Salmonella enterica ssp. enterica, for example from serovar Enteritidis, Dublin, Gallinarum, Typhimurium, Newport, Choleraesuis, Agona, Hadar, Heidelberg, Kentucky, Saintpaul, Virchow, Weltevreden, Javiana, Schwarzengrund, Paratyphi, and Typhi, or from the serovars listed in FIG. 1.

The Salmonella strain which is subject to phoN inactivation as described herein may be an attenuated Salmonella strain, for example a live-attenuated Salmonella strain, for example a live-attenuated Salmonella enterica ssp. enterica serovar Enteritidis His⁻ Ade⁻ strain such as the strain Salmovac SE (Springer et al., 2000, Berl. Münch. Tierärztl. Wschr. 113, 246-252) or a variant thereof.

Inactivation of the phoN gene as described herein may also be applied to authorized live-attenuated Salmonella vaccines. These strains may be transformed into live-attenuated Salmonella ΔphoN marker vaccines without affecting their immunogenicity in order to protect livestock e.g. from Salmonella infection and to simultaneously allow the discrimination of vaccinated from Salmonella infected livestock. This transformation method can be applied for live-attenuated Salmonella vaccine strains which have been prepared by chemical mutagenesis and/or genetic engineering, or for any recombinant Salmonella vaccine approach which uses the live-attenuated Salmonella vaccine strain for example as a carrier to express at least one additional autologous antigen and/or one additional antigen of a heterologous pathogen, or for live-attenuated Salmonella vaccine strain used as a carrier to deliver DNA for vaccination.

The Salmonella strain which is subject to phoN inactivation as described herein being a live-attenuated Salmonella vaccine strain is in particular
(i) a vaccine strain prepared by chemical mutagenesis and/or genetic engineering,
(ii) a recombinant vaccine strain which optionally recombinantly expresses at least one autologous Salmonella antigen and/or at least one heterologous antigen, e.g. an antigen of a heterologous pathogen, or
(iii) a recombinant carrier strain for the delivery of DNA.

A preferred Salmonella strain of the present invention is Salmonella enterica enterica Enteritidis strain CLAB SE404 (DSM 21972), and any strain derived therefrom.

The Salmonella strain of the present invention strain, in particular strain DSM 21972, may be for use in medicine, e.g. in veterinary medicine. The Salmonella strain of the present invention, in particular strain DSM 21972, may be for use as a vaccine. The Salmonella strain of the present invention, in particular strain DSM 21972, may be for use as a live vaccine. The Salmonella strain of the present invention, in particular the vaccine as described herein, more particular strain DSM 21972, may be for use as a vaccine to protect against a Salmonella infection, in particular against an infection with Salmonella enterica ssp. The Salmonella strain of the present invention, in particular strain DSM 21972, may be for use in mammals, e.g. pigs, or birds, e.g. poultry such as chickens.

The Salmonella strain of the present invention strain, in particular strain DSM 21972, may be used for the manufacture of a medicament, e.g. a medicament in veterinary medicine. The Salmonella strain of the present invention, in particular strain DSM 21972, may be used for the manufacture of a vaccine. The Salmonella strain of the present invention, in particular strain DSM 21972, may be used for the manufacture of a live vaccine. The Salmonella strain of the present invention, in particular the vaccine as described herein, more in particular strain DSM 21972, may be used for the manufacture of a vaccine to protect against a Salmonella infection, in particular against an infection with Salmonella enterica ssp. The Salmonella strain of the present invention, in particular strain DSM 21972, may used for the manufacture of a medicament for use in mammals, e.g. pigs, or birds, e.g. poultry such as chickens.

The vaccine of the present invention, in particular strain DSM 21972, may be a vaccine to protect against subsequent Salmonella infection, in particular against a subsequent infection with Salmonella enterica ssp., and to prepare polyvalent recombinant Salmonella carrier vaccines which protect against additional pathogens. The vaccine optionally recombinantly expresses at least one autologous Salmonella antigen and/or at least one heterologous antigen, e.g. an antigen of a heterologous pathogen. The vaccine may be suitable for administration to a mammal, e.g. a pig, or birds, e.g. poultry such as chickens.

Subject of the present invention is a method for prevention or/and treatment of a Salmonella infection comprising administration of a Salmonella strain of the present invention, in particular strain DSM 21972, to a subject in need thereof. The subject may be a mammal, e.g. a pig, or birds, e.g. poultry such as chickens.

A further subject of the present invention is a method of prevention or/and treatment of an infection with a pathogen comprising administration of a Salmonella strain of the present invention to a subject in need thereof, wherein the Salmonella strain of the present invention is prepared to confer protection against this pathogen, for instance a polyvalent Salmonella live vaccine. It is preferred that this Salmonella strain is derived from DSM 21972. The subject may be a mammal, e.g. a pig, or birds, e.g. poultry such as chickens. In particular, the pathogen is different from Salmonella.

Yet another subject of the present invention is a method for production of Salmonella strain as described herein, in particular strain DSM 21972, comprising inactivating the phoN gene of a Salmonella strain.

As indicated above, inactivation of phoN may further reduce the virulence of a live-attenuated Salmonella vaccine strain, but it does not affect the immunogenicity of that transformed Salmonella vaccine strain. Therefore phoN inactivation is a suitable method to produce live-attenuated Salmonella DIVA vaccine strains.

The preparation of a live-attenuated ΔphoN Salmonella DIVA vaccine may start from any live-attenuated Salmonella vaccine strain which is able to protect for instance avian and/or mammal livestock against a Salmonella infection, in particular against an infection with Salmonella enterica ssp. The preparation of DSM 21972 started from a variant of strain Salmovac SE. From such a Salmonella vaccine strain the gene phoN is inactivated as described herein by applying genetic engineering. After completion of the inactivation of phoN, the individual Salmonella variant strains have to be analyzed in order to evaluate differences which appear in relation to the parental Salmonella vaccine strain. The ΔphoN Salmonella variant strain are preferably essentially identical in the biological characteristics except phoN from the parental Salmonella vaccine strain. If the ΔphoN Salmonella variant strain differs from the parental Salmonella vaccine strain in one or more characteristics not related to phoN, these alterations may be permanent and, most importantly, may essentially maintain the original immunogenic character of the parental strain which provokes in appropriately vaccinated animals a protective immune response against subsequent Salmonella infection.

Preferentially, phoN is inactivated irreversibly by deletion of the nucleic acid fragment encoding the gene phoN by means of genetic engineering. The inactivation process may also include the deletion of accessory nucleic acids from which the transcription of phoN is initiated. The deletion of phoN may be performed in such a manner which suppresses the appearance of novel transcripts. Genetic engineering encompasses any processes of homologous recombination which enable the deletion of the gene phoN in the genome of Salmonellae as indicated herein. Preferably, the process of homologous recombination should enable the precise deletion of the gene phoN whilst the rest of the genome remained unchanged, which is designated herein as “clean” deletion process. Furthermore a process of homologous recombination is preferred which is driven by a temporarily co-expressed recombinase, such as the phage λ Red recombinase system as described by Datsenko and Wanner.

The Salmonella strain of the present invention may have a reduced motility, e.g. by at least 30%, at least 50% or at least 90%, or/and a reduced biofilm forming capability, e.g. by at least 30%, at least 50% or at least 90%.

As indicated above, the ΔphoN Salmonella strain, in particular the live-attenuated ΔphoN Salmonella strain, more particular DSM 21972, may be a marker vaccine. Live-attenuated ΔphoN Salmonella marker vaccine strains such as DSM 21972 enable the Differentiation of infected from Vaccinated Animals (DIVA) by serological and/or supplementary bacteriological means.

Yet another subject of the present invention is the use of a polypeptide comprising a Salmonella PhoN polypeptide or/and an immunogenic fragment thereof as a serological marker antigen. The serological marker antigen may be any antigen derived from the PhoN polypeptide. The phoN antigen may be a polypeptide comprising the full-length PhoN polypeptide, or comprising any immunogenic portion thereof which has a length of preferably at least 10, more preferably at least 20 and even more preferably at least 30 amino acids. The PhoN polypeptide may be a polypeptide comprising a sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:18, SEQ ID NO:19, and SEQ ID NO:20, or a polypeptide comprising a sequence which is at least 70%, at least 80%, at least 90%, at least 95%, at least 98%, or at least 99% identical to a sequence selected from SEQ ID NO:1, SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, and SEQ ID NO:20, wherein % identity is calculated relative to the full length of the selected sequence. The immunogenic portion may have a sequence having at least 70%, at least 80%, at least 90%, at least 95%, at least 98%, or at least 99% identical amino acid positions to the sequence of a PhoN polypeptide described herein, wherein % identity is calculated relative to the length of the immunogenic portion. Identity as described herein may be calculated by common algorithms such as BLAST or FASTA. A preferred sequence is SEQ ID NO: 1 derived from strain CLAB_SE 360 (see FIG. 1).

The serological marker antigen as described herein may be used for the differentiation of infected from vaccinated animal (DIVA) after vaccination with the Salmonella strain of the present invention. A serological marker antigen derived from SEQ ID NO:1 may in particular be used for differentiation of an infected animal from an animal vaccinated with strain DSM 21972.

The phoN antigen of the present invention may be prepared by recombinant expression of a phoN nucleic acid, particularly in a recombinant host cell such as E. coli. The phoN antigen may be provided as a frozen stock or as a lyophilisate, optionally together with appropriate preservatives.

Yet another subject of the present invention is a serological test system for detecting antibodies directed against a Salmonella phoN antigen, comprising at least one recombinant Salmonella phoN antigen or any immunogenic portion thereof, and optionally further test components. The serological test system may be capable of discrimination of Salmonella infected animals from animals vaccinated by a Salmonella strain of the present invention, in particular a live-attenuated ΔphoN Salmonella DIVA vaccine, such as CLAB SE404 (DSM 21972). The at least one phoN antigen is an antigen as described herein. The Salmonella phoN antigen preferably comprises the sequence SEQ ID NO:1 or a sequence which is at least 70%, at least 80%, at least 90%, at least 95%, at least 98%, or at least 99% identical to SEQ ID NO:1, wherein identity is calculated relative to the length of SEQ ID NO:1.

In the serological test system, the antigen serves as a target antigen which specifically detects antibodies in the serum of animals suspected of being infected with Salmonella or may become infected with Salmonella after vaccination. No antibodies are detected by the PhoN target antigen if the serum of animals is used which have been successfully immunized by a Salmonella strain of the present invention, in particular a live-attenuated ΔphoN Salmonella DIVA vaccine. Detection may be performed in the ELISA format.

Optionally an additional Salmonella specific antigen is used in the serological test system, such as Salmonella LPS, which detects antibodies induced by both wildtype Salmonella and the Salmonella vaccine strain of the present invention, in particular the live-attenuated ΔphoN Salmonella DIVA vaccine.

Yet another subject of the present invention is a phoN nucleic acid encoding a Salmonella antigen as described herein. The phoN nucleic acid comprises a sequence encoding a PhoN polypeptide or a fragment thereof as described herein, for instance a polypeptide comprising a sequence selected from SEQ ID NO:1, SEQ ID NO:2, SEQ ID NO:3, SEQ ID NO:4, SEQ ID NO: 5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO:16, SEQ ID NO:17, SEQ ID NO:18, SEQ ID NO:19, and SEQ ID NO:20, or a polypeptide comprising a sequence which is at least 70%, at least 80%, at least 90%, at least 95%, at least 98%, or at least 99% identical to said sequence, wherein identity is calculated relative to the length of said sequence. The nucleic acid may code for any immunogenic fragment of a PhoN polypeptide as described herein.

The phoN nucleic acid may be used for the manufacture of a recombinant Salmonella phoN antigen or any immunogenic portion thereof, e.g. in a host such as E. coli by using an appropriate recombinant DNA vector. The phoN antigen may be an antigen as described herein. The phoN antigen may comprise the sequence SEQ ID NO:1, or a sequence which is at least 70%, at least 80%, at least 90%, at least 95%, at least 98%, or at least 99% identical to SEQ ID NO:1, or any immunogenic portion thereof, as described herein. Identity is calculated relative to the length of SEQ ID NO:1.

A further subject of the present invention concerns a bacteriological test system for the differentiation of wildtype Salmonella ssp. and a Salmonella strain of the present invention, such as a live-attenuated ΔphoN Salmonella DIVA vaccines. This bacteriological test system is completing the official standard methods which regulate the detection of Salmonella in biological samples.

The inventive bacteriological test system clarifies whenever Salmonella colonies appear in the standard detection methods whether these are wild-type Salmonella or Salmonella strain of the present invention, such as a live-attenuated ΔphoN Salmonella. At least one suspicious Salmonella colony is analyzed in order to evaluate whether the genome of the individual Salmonella colonies contains the gene phoN or a fragment thereof. Analysis may be performed by a PCR method, or/and by detection of PhoN activity on and a PhoN gene product. A representative number of suspicious Salmonella colonies may be analyzed, for instance at least 5, at least 10, at least 15, or at least 20 colonies.

PhoN activity can be measured by transferring aliquots of single colonies into separate vials, such as a well of microtiter plate, which contain a PhoN specific substrate mix where PhoN generates a chromogenic product that can be detected by visual control or by an appropriate device. PhoN is localized in the periplasmic space of Salmonella ssp which allows the use of intact cells for determination of PhoN activity. According to the test system wildtype Salmonella show PhoN activity by generating a chromogenic product whereas live-attenuated ΔphoN Salmonella do not. A suitable substrate may be BCIP.

The Salmonella strain of the present invention may be detected by a test according to ISO 6579. ISO 6579 is included herein by reference. By a test according to ISO 6579, PhoN activity may be detected by the chromogenic substrate BCIP.

The invention is further illustrated by the following Figures, Examples and the sequence listing.

### FIGURE LEGENDS

FIG. 1: Similarities of PhoN proteins between various Salmonella enterica enterica serovars.

FIG. 2: The genomic regions of phoN of Salmonella enterica enterica serovars.

FIG. 3 illustrates the strategy for the preparation of phoN deletion mutant of live-attenuated Salmonella enterica enterica Enteritidis vaccine strain Salmovac SE using replacement recombination as described by Datsenko and Wanner. The genomic region of S. Enteritidis strain P125109 is exemplarily shown encompassing the phoN locus which is identical to S. Enteritidis vaccine strain Salmovac SE. The genomic region starts at nucleotide (nt) 4.419.410 of the sequence NC_{—}011294.1 which is deposited at the NCBI (National Center for Biotechnology Information) data base (http://www.ncbi.nlm.nih.gov/genomes). The orientation and length of any open-reading-frame (ORF) is indicated by arrows. Annotated ORFs are illustrated by black arrows and the others by striped arrows. For phoN, two promoters (P1, P2) have been predicted which are illustrated by open arrows.

FIG. 3A exemplarily illustrates the boundary (solid vertical lines) of the deletion region which comprises the gene phoN. FIG. 3B illustrates the ORFs within the original genomic region after replacement of phoN. Three of the original ORFs (ORF1-3) become extended by the recombination process (open arrow).

FIG. 4 illustrates reverse transcriptase PCR (RT-PCR) analyses within the genomic region of a phoN mutant strain prepared from S. Enteritidis vaccine strain Salmovac SE by replacement recombination as outlined in FIG. 3.

In section A, data of RT-PCR analyses of the ORF designated as putative acetyltransferase (black arrow) are illustrated. The analyses revealed a transcript (mRNA) that encompasses the putative acetyltransferase and the following ORFs 1-3. The RT-PCR analyses further revealed that the transcript extends into the replacement fragment but, surprisingly, does not pass that fragment.

In the upper right frame, the primary structure of the replacement fragment and the included FRT-site is shown. The FRT-site forms a dyad structure of high stability (−17 kcal/mol). It is suggested that the FRT-site actually acts as a transcriptional terminator which suppresses the expression of the ORFs 1-3 beyond the replacement fragment.

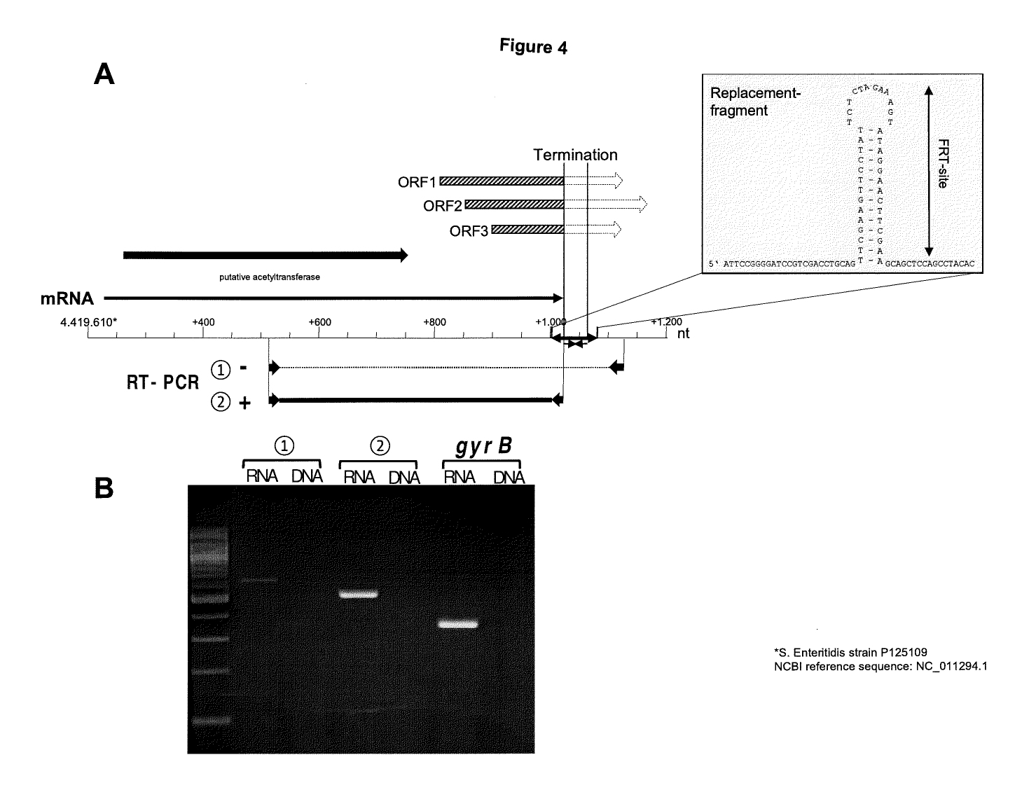

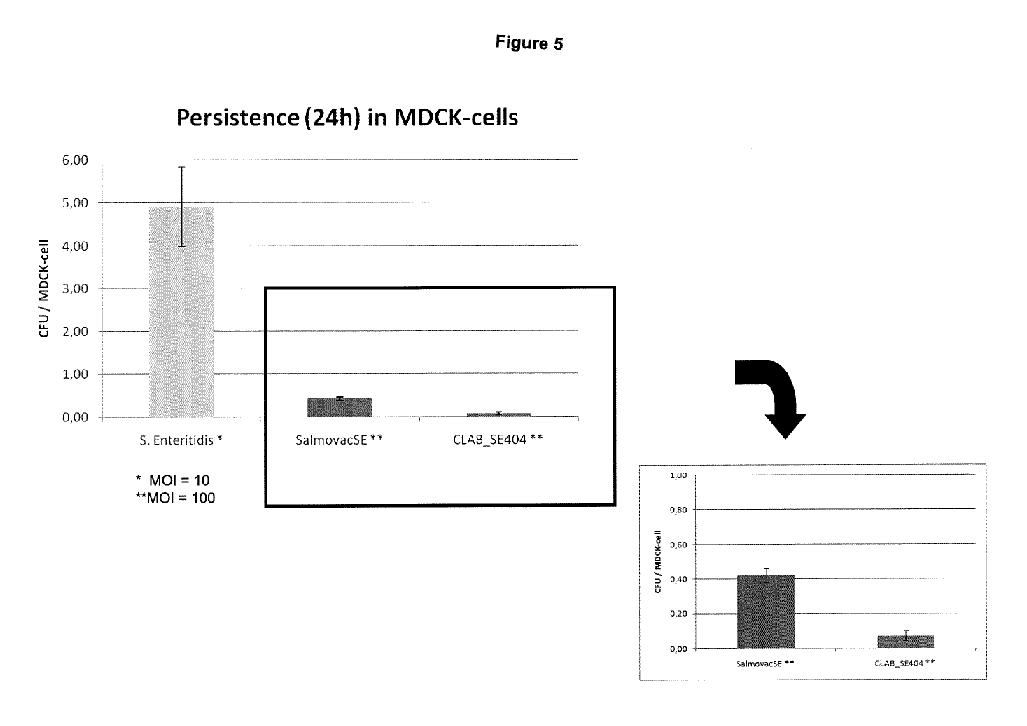

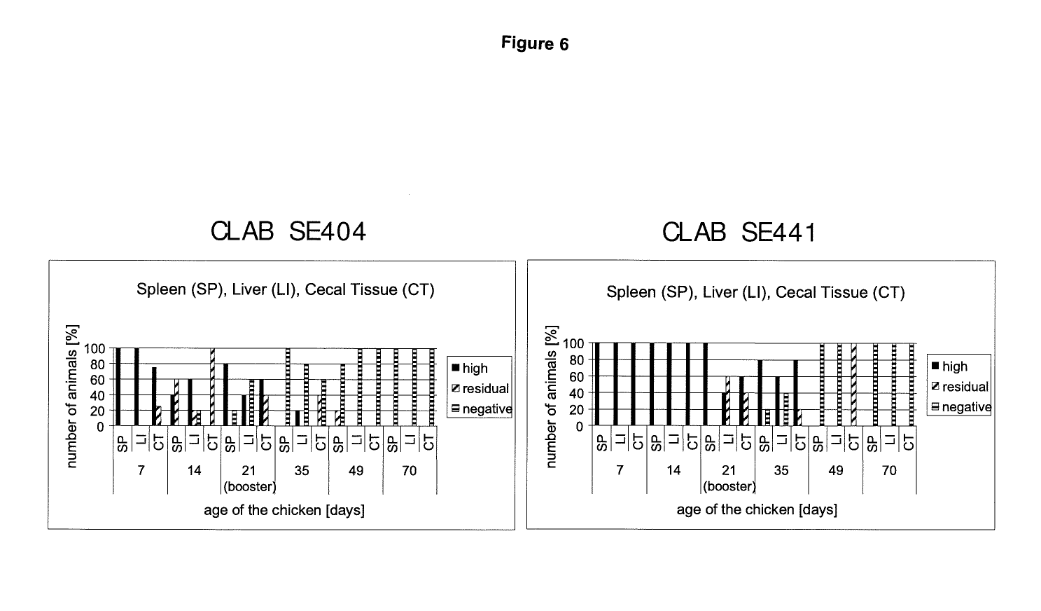

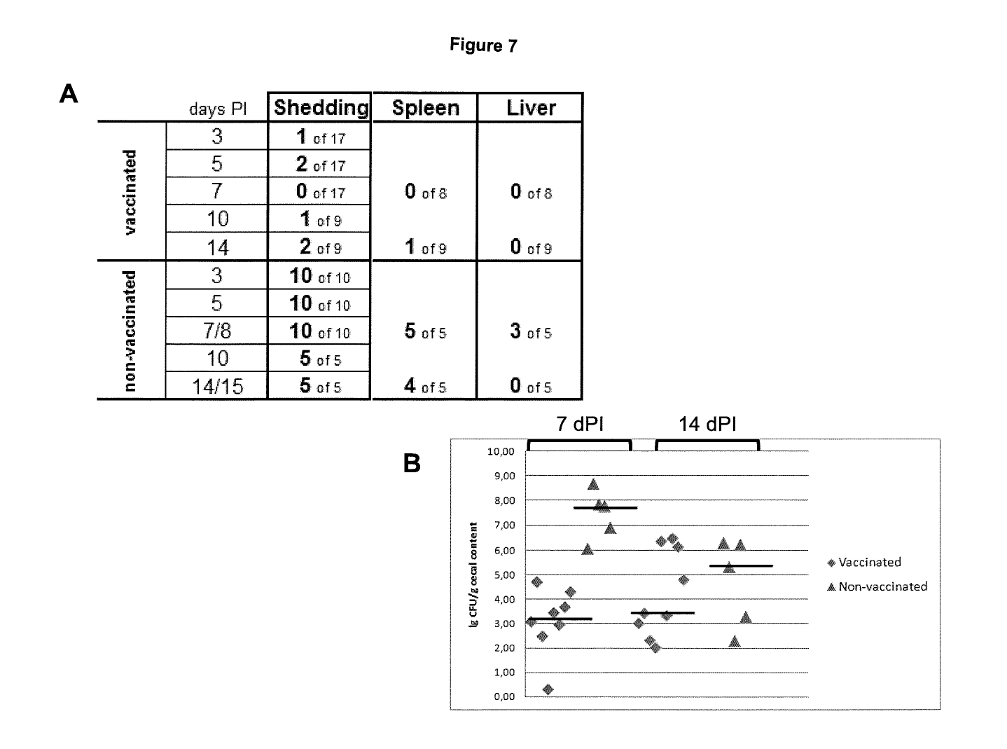

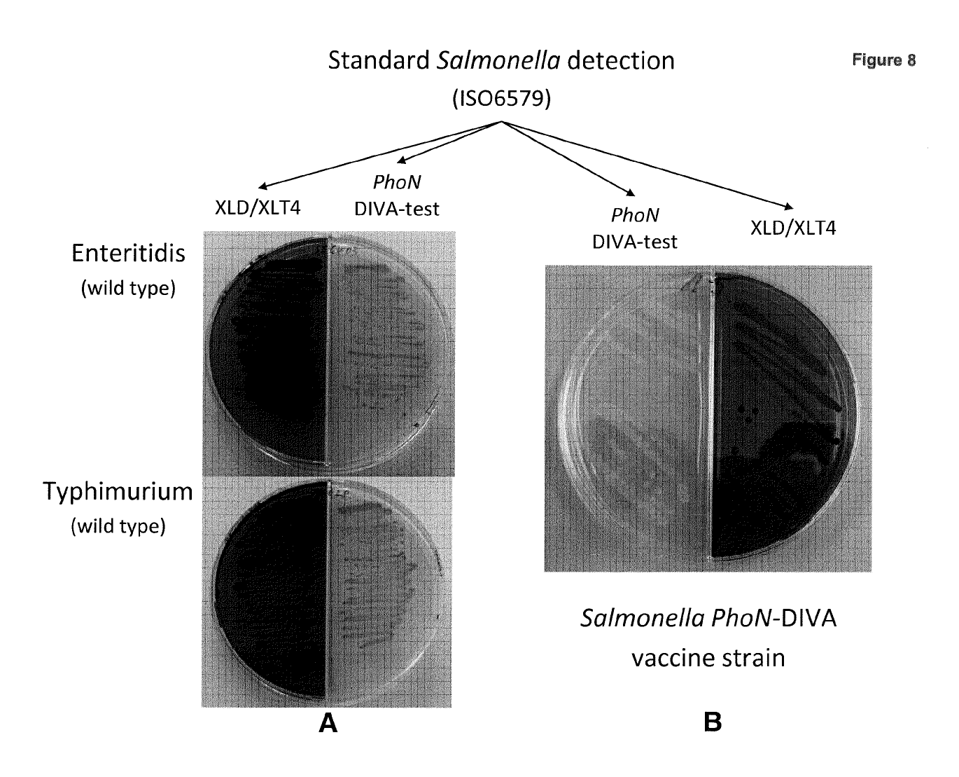

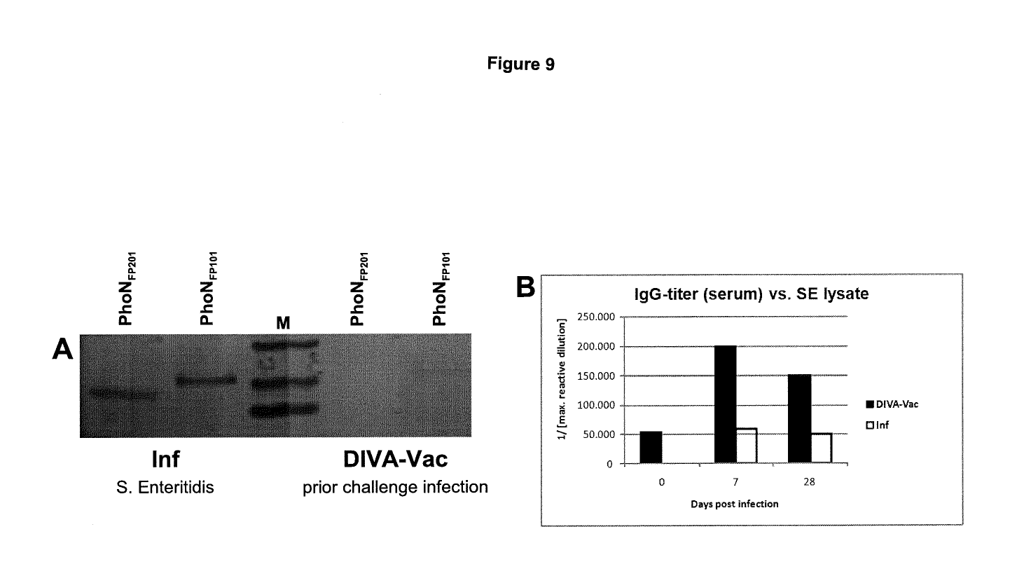

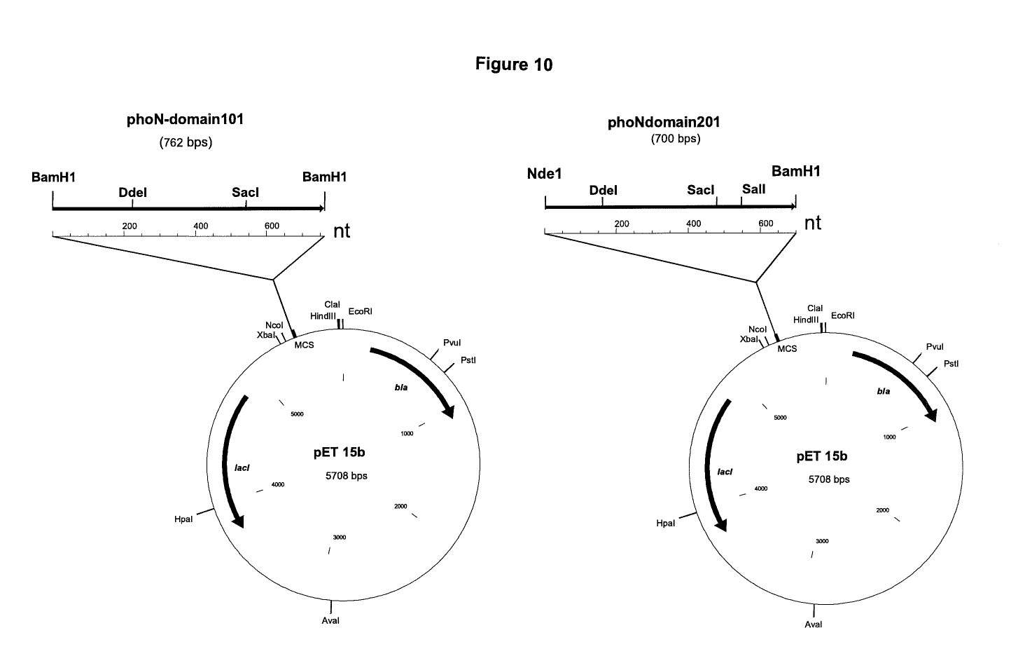

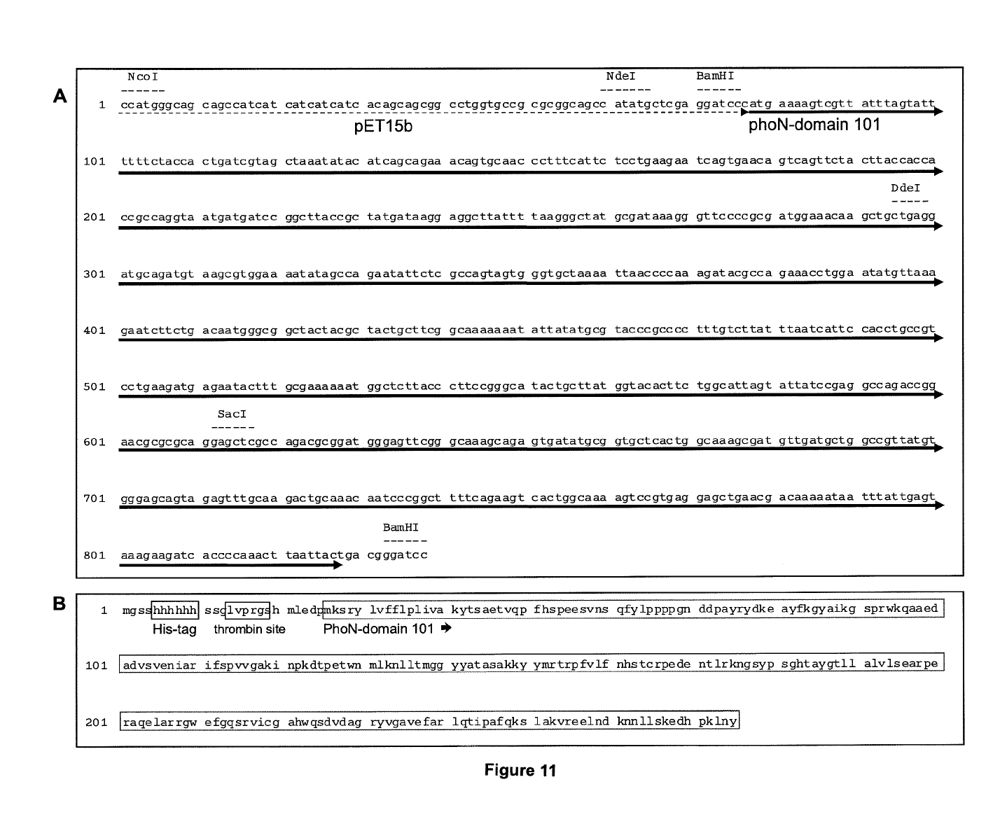

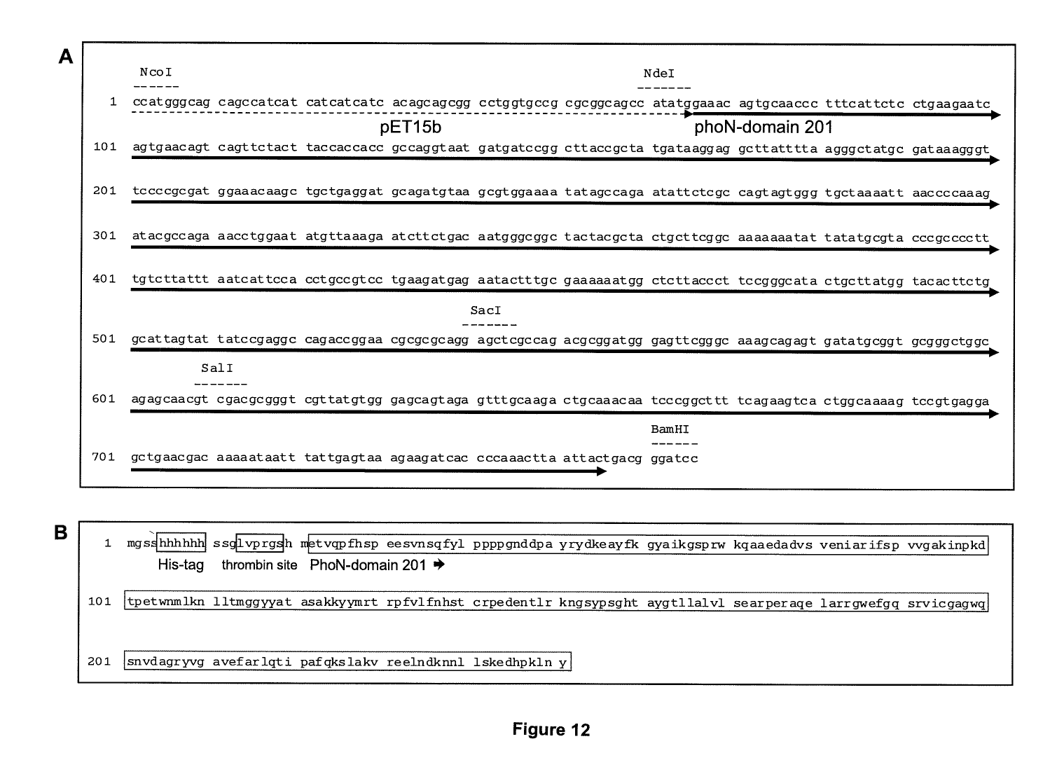

The primers for the RT-PCR are conceived to detect transcripts which either pass through the replacement fragment (1) or which cover the region in front of the FRT-site (2). FIG. 4B shows agarose gel electrophoresis of the RT-PCR products obtained from RNA preparations of S. Enteritidis ΔphoN vaccine strain which have been treated with reverse transcriptase (RNA) or remained untreated (DNA) before PCR. The data reveal pure RNA probes. As a control, the RT-PCR of the gene gyrB is presented. As already indicated, the transcript beyond the replacement fragment is almost abolished (lane 1) which is in contrast to the transcript that ends in front of the FRT-site (lane 2).

FIG. 5

The virulence of wild type S. Enteritidis strain is compared with live-attenuated S. Enteritidis vaccine strain Salmovac SE and ΔphoN mutant strain CLAB_SE404. The relative virulence of the various Salmonella strains was assessed by determining the survival rate of Salmonella in MDCK cells within a 24 hours period. The cells were infected with wild type S. Enteritidis strain at an MOI of 10. Both live-attenuated S. Enteritidis vaccine strains were administered at an MOI of 100. After an infection period of 2 hours, excess bacteria in the culture medium were removed by washing. Additionally gentamycin [25 μg/mL] was added in order to suppress extra-cellular Salmonella. After 24 h cultivation of infected cells under appropriate conditions, the number of intra-cellular Salmonella was determined by lysis of the MDCK cells in PBS containing 1% Triton-X100.

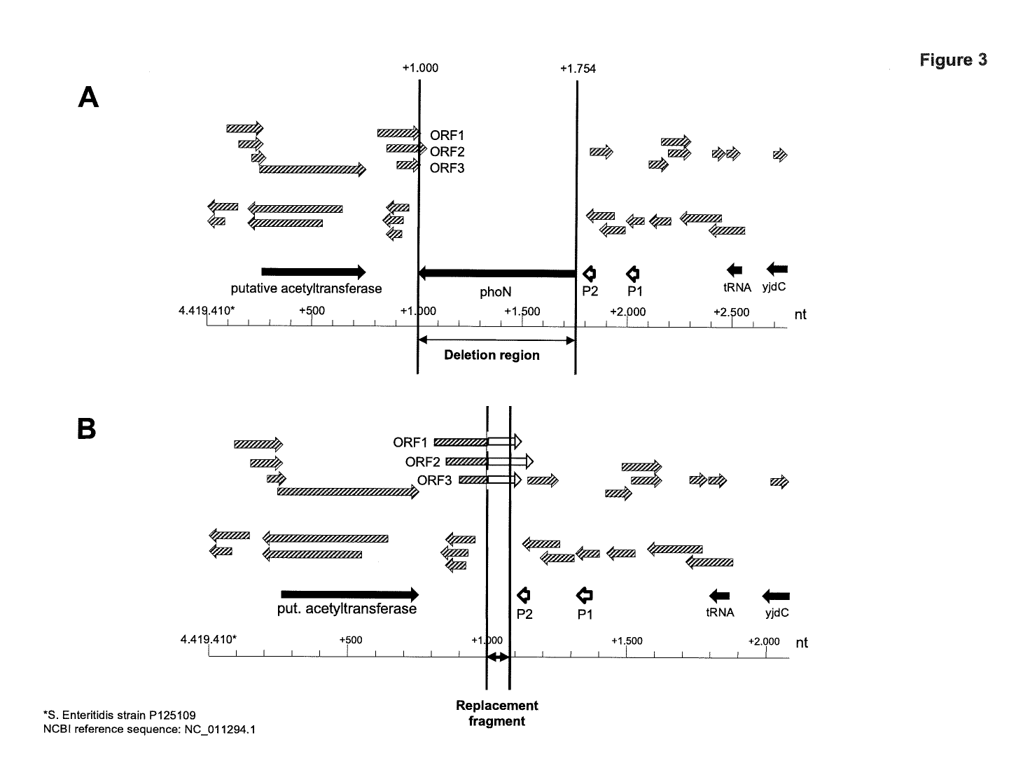

The data clearly show the strongly reduced persistence of both live-attenuated S. Enteritidis vaccine strains in MDCK cells compared to wild type S. Enteritidis isolate. Surprisingly, the data revealed different survival rates for both Salmonella vaccine strains in MDCK cells. CLAB_SE404 is a non-motile variant of different ΔphoN mutant clones which have been prepared from live-attenuated S. Enteritidis vaccine strain Salmovac SE as described in the legend to FIG. 3. The data indicate reduced virulence of CLAB_SE404 compared to the progenitor strain Salmovac SE.

FIG. 6

The virulence of two ΔphoN S. Enteritidis vaccine strains was further evaluated in White Leghorn chicken. The animals were vaccinated orally at the age of 1 and 21 days with 1-2×10⁸ CFU of either CLAB_SE404 or CLAB_SE441. CLAB_SE441 is a motile variant of S. Enteritidis ΔphoN [ade/his]. In the contrast CLAB_SE404 is a non-motile variant of S. Enteritidis ΔphoN [ade/his].

At different time after vaccination, 5 animals were sacrificed and the number of bacteria was determined in the various tissues of the vaccinated animals. High colonization value indicates that Salmonella were detected by direct cultivation on selective XLT-4 agar plates. Residual colonization was determined after enrichment cultivation in peptone water and subsequent incubation on MSRV agar plates as described in ISO6579.

The data confirm the differences in virulence between both ΔphoN S. Enteritidis vaccine strains. CLAB_SE404 disappears much more rapidly from the tissues of vaccinated chicks compared to CLAB_SE441.

FIG. 7

White Leghorn chicks which were vaccinated twice with CLAB_SE404, as described in the legend to FIG. 6, received an oral challenge infection with 1-2×10⁸ CFU of wild type S. Enteritidis (SE 147N). The challenge infection was administered 21 days after booster vaccination and was analyzed for further 14 days. As a control, naïve White Leghorn chicks were infected at the same age of 42 days.

At different times, the shedding and tissue colonization of Salmonella within both animals groups was determined and is summarized in FIG. 7A. The data reveal clear protection of the vaccinated animals. Thus the number of animals which shed wild type S. Enteritidis is clearly reduced in the vaccinated group. Furthermore, the number of animals with Salmonella in spleen and liver is again low in the vaccinated compared to the untreated control group. Vaccinated animals had even significantly reduced numbers of wild type Salmonella in cecal content than naïve animals (FIG. 7B). The data clearly demonstrate the protective property of CLAB_SE404, the non-motile variant of Salmovac SE ΔphoN vaccine strain.

FIG. 8

Approval of the Bacteriological PhoN-DIVA Test which is Used in Parallel to Standard Salmonella Detection Test According to ISO6579.

FIG. 8A exemplarily illustrates parallel plating of wild-type Salmonella Typhimurium and Enteritidis, respectively, on XLT4 agar plates and on LB-agar plates supplemented with the chromogenic phosphatase substrate BCIP [80 μg/mL]. Wild-type Salmonellae Typhimurium and Enteritidis generally appear on XLD/XLT4 agar plates as black colored colonies and on BCIP LB-agar plates (PhoN-DIVA test) as blue-green colored colonies.

In FIG. 8B the parallel plating of the live attenuated Salmonella Enteritidis ΔphoN vaccine strain CLAB_SE404 is shown. The ΔphoN Salmonella vaccine strain appears as black-colored colonies on Salmonella specific XLD/XLT4 agar plates but remained uncolored on BCIP LB-agar plates. This finding is surprising, since it is known that Salmonella expresses several enzymes with phosphatase activity. It is concluded that inactivation of the phoN gene is sufficient to prevent the chromogenic phosphate reaction described herein.

The outlined testing allows clear differentiation of infected from vaccinated animals within the standard Salmonella test set-up using live-attenuated phoN-DIVA S. Entertitidis vaccine strain CLAB_SE404.

FIG. 9

### PhoN-DIVA Antibody Test in White Leghorn Chicken.

Specificity of PhoN-DIVA antibody test as approved by immunoblot analysis using sera from Salmonella-infected chicks (Inf) and sera from chicks immunized with live-attenuated S. Enteritidis phoN-DIVA vaccine strain CLAB_SE404 (Vac).

The blots of graph A were prepared with two recombinant variants of Salmonella PhoN, PhoN_{FP101} and PhoN_{FP201}. The purified samples have been separated on 10% Schagger-Jagow PAGE prior blotting. PhoN_{FP101} covers the primary structure of the original PhoN. PhoN_{FP201} has truncated NH2-terminus and amino acids substitutions within the active center of PhoN yielding an inactive PhoN. The immunoblot analyses show IgG-specific immune response using appropriate alkaline phosphatase-conjugated antisera. Molecular weight markers are shown in lane M, corresponding to 43, 34 and 26 kDa, respectively.

The PhoN-DIVA antibody test displayed in FIG. 9A has been carried out with sera from chicks immunized twice with live-attenuated S. Enteritidis phoN-DIVA vaccine strain CLAB_SE404 (Vac) and sera from naïve animals infected with virulent S. Enteritidis strain SE 147N (Inf). The antibodies of the Inf-group bind to both variant PhoN antigens. The Vac-group does not produce PhoN-specific antibodies.

In FIG. 9B, the IgG-titer against S. Enteritidis lysate is illustrated as determined in an ELISA using sera of both animal groups at different times of infection. In the DIVA-Vac group, IgG-titer was determined before (0) and after challenge infection, at days 7 and 28 post infection. In the Inf group the IgG-titer was determined in the same manner.

FIG. 10

Maps of the expression plasmids used for the preparation of the fusion proteins PhoN_{FP101} (left-hand map) and PhoN_{FP201} (right-hand map).

FIG. 11

Sequence of the gene (A) and the encoded fusion protein (B) of PhoN_{FP101}.

FIG. 12

Sequence of the gene (A) and the encoded fusion protein (B) of PhoN_{FP201}.

### EXAMPLE 1

The similarities of the protein PhoN has been assessed within the various Salmonella enterica enterica serovars by BLAST 2.2.18 using the database “nr” via the Internet portal of the National Center for Biotechnology Information (NCBI). The amino acid sequence of the protein PhoN of the Salmonella Enteritidis strain CLAB_SE360 has been used as query sequence (SEQ ID NO:1). CLAB_SE360 is a variant of the vaccine strain Salmovac SE (Springer et al., 2000, Berl. Münch. Tierärztl. Wschr. 113, 246-252). Non-identical amino acids within the primary structure of the various PhoN proteins are highlighted by color. The data reveal that the primary structures of the PhoN protein within the various Salmonella serovars have an identity which is >96%.

In contrast, the primary structures of the PhoN protein within the non-Salmonella bacteria have low relationship (identity <40%) with the PhoN protein of Salmonella enterica enterica serovars. The most related PhoN amino acid sequences of non-Salmonella bacteria are shown. Only identical amino acids are displayed. Variant (−), additional (+) or missing (Δ) amino acids are indicated. The full sequences are given in the sequence listing (SEQ ID NO:21 and 22).

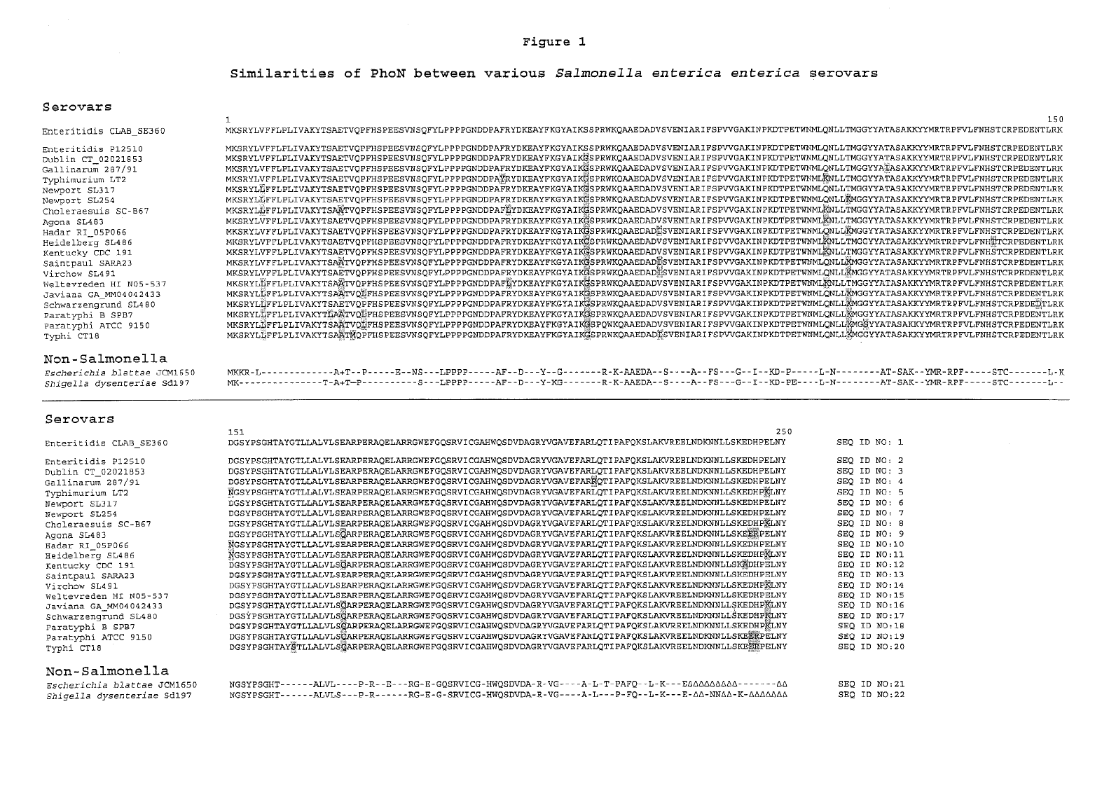

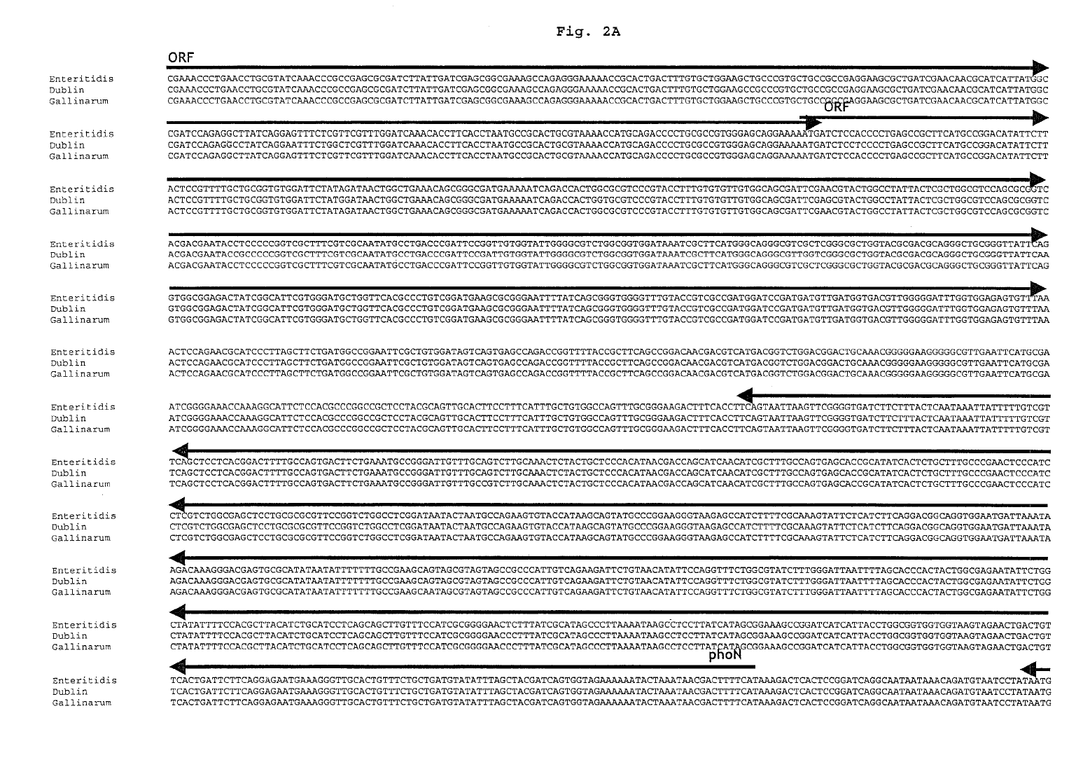

The results are summarized in FIG. 1.

### EXAMPLE 2

Description of the phoN genomic regions of various Salmonella enterica enterica serovars comprising the gene phoN and the flanking regions which are of relevance for the preparation of ΔphoN Salmonella mutants by genetic engineering, such as described by T. Shigaki and K. D. Hirschi (Anal. Biochem. 2001, 298:118-120) or by K. A. Datsenko and B. L. Wanner (PNAS 2000, 97:6640-6645).

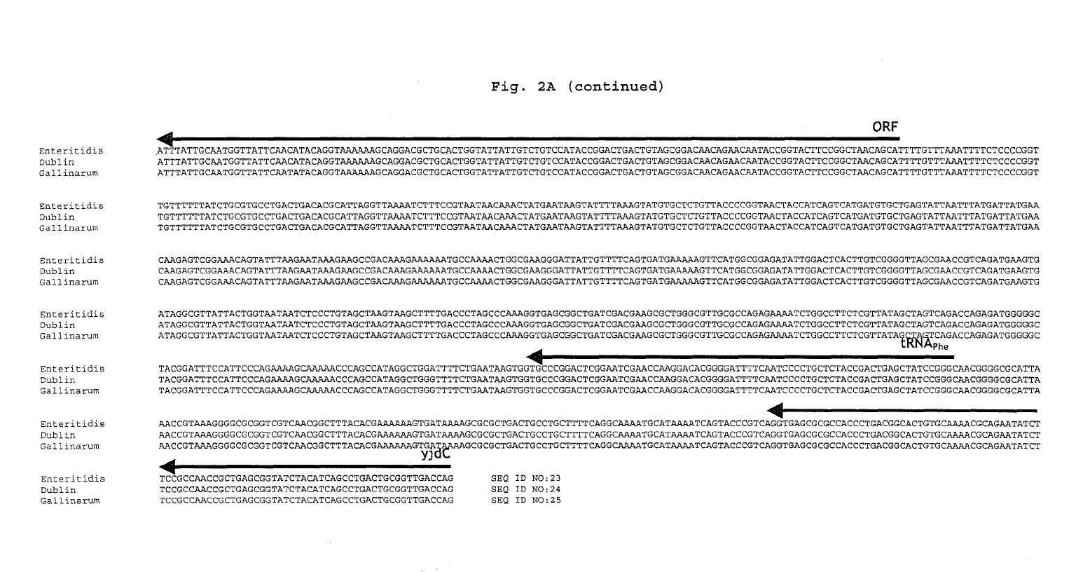

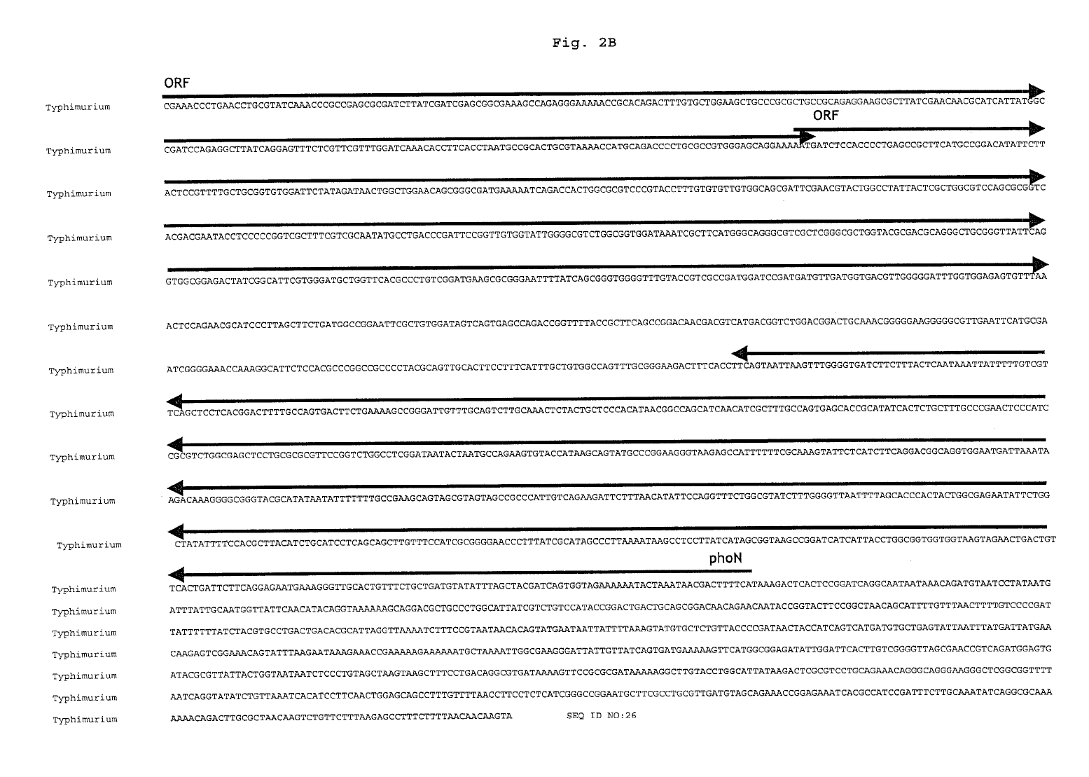

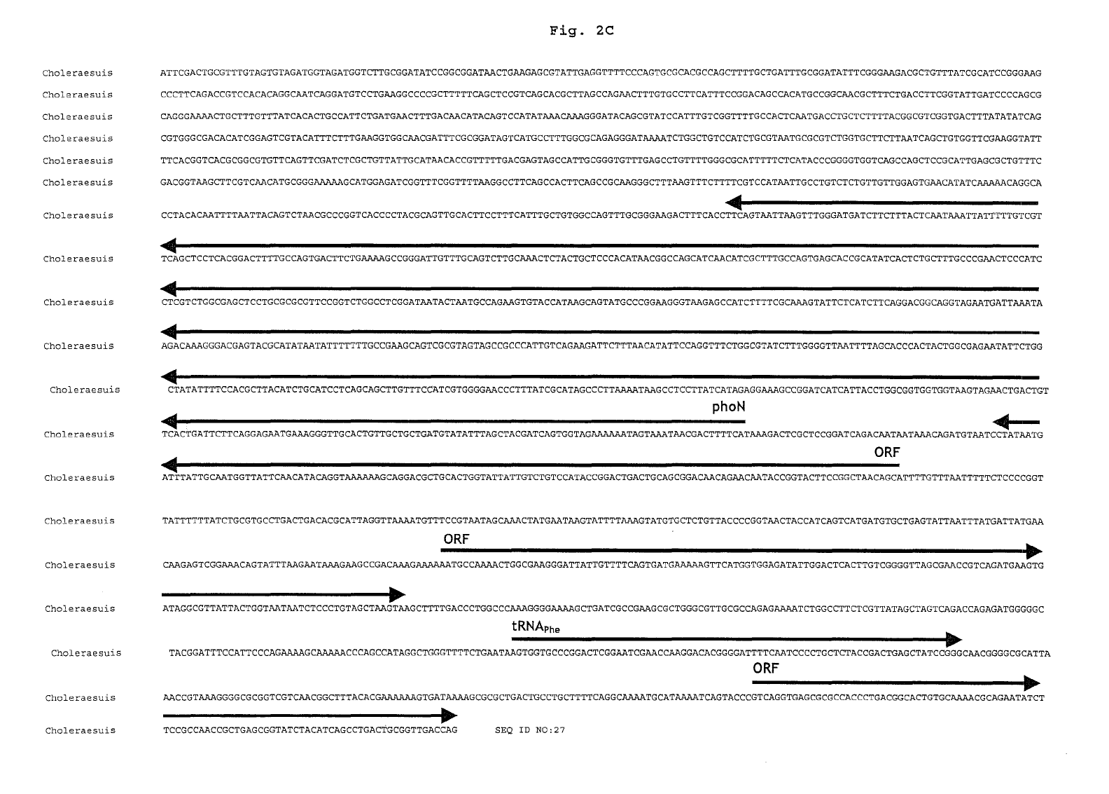

The phoN genomic regions are shown in FIG. 2 as follows:
FIG. 2A S. Enteritidis str. P125109 (excerpt from RefSeq NC011294.1) S. Dublin str. CT_{—}02021853 (excerpt from RefSeq NC011205.1)
   S. Gallinarum str. 287/91 (excerpt from RefSeq NC011274.1)
FIG. 2B S. Typhimurium str. LT2 (excerpt from RefSeq NC003197.1)
FIG. 2C S. Choleraesuis str. SC-B67 (excerpt from RefSeq NC006905.1)

### EXAMPLE 3

In this Example, the gene phoN has been removed by genetic engineering from the genome of a Salmonella Enteritidis strain which originally derived from a variant of the live-attenuated Salmonella Enteritidis vaccine strain Salmovac SE. More detailed analyses surprisingly showed differences in the population of the ΔphoN Salmonella Enteritidis vaccine strain variants. Some few ΔphoN vaccine strain variants have motile activity which is at least 30% of the progenitor strain and the other ΔphoN vaccine strain variants. In addition, the ΔphoN vaccine strain variants of reduced motility have reduced capability to form biofilm which, again, is different from the progenitor strain and the other ΔphoN variants.

Besides the indicated differences, all ΔphoN Salmonella Enteritidis variants investigated are auxotroph for adenine and histidine and express complex lipopolysaccharide (LPS). They do not contain any of the known virulence plasmids. Thus, all ΔphoN Salmonella Enteritidis variants include the relevant characteristics of Salmovac SE which has been approved for vaccination laying hens in Germany.

A single variant, CLAB SE404, has been selected from the population of the ΔphoN Salmonella Enteritidis variants which have reduced motility and biofilm capability. Further analyses of CLAB SE404 revealed that these additionally identified characteristics are stable beyond 150 generations.

The strain CLAB SE404 has been deposited under the Budapest Treaty on 10 Nov. 2008 at the DSMZ-Deutsche Sammlung von Mikroorganismen und Zellkulturen GmbH, Inhoffenstraβe 7 B, D-38124 Braunschweig under deposit number DSM 21972.

In following analyses the virulence and efficacy of CLAB SE404 has been compared with the motile ΔphoN Salmonella Enteritidis vaccine strain variants and the original vaccine strain.

The virulence of the Salmonella vaccine strains has been assessed by in vitro and in vivo experiments. Quantitative infection of MDCK-cells clearly showed that CLAB SE404 is less virulent in MDCK-cells than the progenitor strain and the motile ΔphoN Salmonella Enteritidis variants.

One-day-old chickens were orally inoculated with either CLAB SE404 or a single variant of the motile ΔphoN Salmonella Enteritidis group. Afterwards the presence of Salmonella in the various tissues of the vaccinated animals was determined by bacteriological means. The data reveal similar amount of Salmonella in liver and spleen in both animals groups 7 days after vaccination. Thereafter the amount of CLAB SE404 declines in the liver and in the spleen but it remained high in the organs of the animals which received the motile ΔphoN Salmonella Enteritidis variant strain. In the cecal tissue similar results were obtained. The data clearly indicate that CLAB SE404 is less virulent than the motile ΔphoN Salmonella Enteritidis variant. CLAB SE404 disappeared from the tissues and the excrements of vaccinated chickens much earlier than the motile ΔphoN Salmonella Enteritidis variant.

The immunogenicity of the ΔphoN Salmonella Enteritidis strains has been determined in chickens which were orally vaccinated by either CLAB SE404 or the motile ΔphoN Salmonella Enteritidis variant at the age of 1 and 21 days. The humoral and mucosal immune response of both animal groups was assessed by ELISA using Salmonella antigens. The data revealed that both vaccine strains provoked in orally vaccinated chickens a humoral and mucosal immune response which remained high for more than 50 days after the last vaccination. Thus, CLAB SE404 has similar immunogenic efficacy as the motile ΔphoN Salmonella Enteritidis variant.

Finally, it could be shown that chickens which were orally vaccinated by either CLAB SE404 or the motile ΔphoN Salmonella Enteritidis variant at the age of 1 and 21 days resist challenge infection with virulent Salmonella enterica Enteritidis isolate. Thus, CLAB SE404 has similar protective efficacy as the motile ΔphoN Salmonella Enteritidis variant.

In summary, CLAB SE404 has been identified as a novel live-attenuated Salmonella vaccine for poultry with low shedding and high efficacy which further enables differentiation of infected from vaccinated animals.

### EXAMPLE 4

### Preparation of Recombinant PhoN Protein for PhoN-DIVA Antibody Test

PhoN-DIVA antibody test is preferentially prepared on the basis of recombinant PhoN. Previous immunoblot analyses with purified recombinant PhoN clearly reveal that animals vaccinated with live-attenuated Salmonella phoN-DIVA vaccine strain do not generate antibodies which bind to the whole protein. It was surprising that no cross-reactive antibodies do exist within sera of animals immunized with phoN-DIVA Salmonella vaccine strain, despite the complexity of the protein PhoN and the broad-spectrum reactivity of the humoral immune response.

The recombinant PhoN for PhoN-DIVA antibody test may be prepared with high purity. Any impurities may be detected by the broad-spectrum antibodies of livestock immunized with PhoN-DIVA Salmonella vaccine and thus cause false results in the PhoN-DIVA antibody test.

The preparation of pure PhoN may be facilitated by extending the primary structure of PhoN with an extra polypeptide fragment that allows highly stringent purification conditions. Preferentially, a series of histidine (His-tag) is introduced either at the amino terminus or at the carboxy terminus of PhoN. Furthermore, another protein domain may be introduced between the His-tag and the PhoN structure which is specifically recognized by an endopeptidase, e.g. thrombin, which allows the removal of the His-tag. The analyses reveal that such a fusion protein of PhoN which comprises the His-tag and thrombin-site does not interfere with sera of animals vaccinated with live-attenuated Salmonella DIVA-vaccine strain as demonstrated in the previous analyses.

The fusion proteins of PhoN can be prepared in Escherichia coli by conventional expression vector, e.g. pET15b and by applying standard purification protocol as indicated by the supplier, e.g. Novagen. FIGS. 10-12 disclose the maps of the used expression plasmids, the DNA and the amino acid sequences of the exemplarily prepared PhoN fusion proteins, PhoN_{FP101} and PhoN_{FP201}.

### PhoN-DIVA Antibody Test

PhoN-DIVA antibody test is applied whenever chicken become vaccinated with live-attenuated Salmonella phoN-DIVA vaccine strain. In this Example, the serological PhoN-DIVA test system comprises the recombinant protein PhoN, e.g. PhoN_{FP101} or PhoN_{FP201}, and another Salmonella specific antigen. Preferentially, the Salmonella specific antigen is provided by a commercial test kit which detects Salmonella specific antibodies in chicken, e.g. provided by Idexx Laboratories Inc. (Flockchek) or by Labor Diagnostik GmbH Leipzig (Flocktype Salmonella) or by Biochek and other companies. This test set-up proves whether Salmonella specific immune response appears after vaccination. Additionally, it proves that no wild-type Salmonella was involved which is indicated by negative reaction with recombinant protein PhoN in the PhoN-DIVA antibody test.

Generally, the commercial test kits for the detection of Salmonella specific antibodies are prepared as an indirect ELISA in microtiter plates. Depending on the provider, the Salmonella-specific antigen or antigen mix is already bound to the vials of the microtiter plate or must be coupled later on using an aliquot of a stock solution.

The purified PhoN, e.g. PhoN_{FP201}, is preferentially provided as a frozen stock solution or as a lyophilisate with appropriate preservative agents. In this Example, the ELISA for the detection of PhoN-specific antibodies is performed according to standard protocol as described elsewhere, e.g. by E. Harlow and D. Lane in Antibodies. A Laboratory Manual.

1. Salmonella enterica ssp. enterica serovar Enteritidis strain DSM 21972 or any strain derived therefrom.

2. The strain of claim 1 for use in medicine, e.g. in veterinary medicine.

3. The strain of claim 2 for use as a vaccine.

4. The strain of claim 3 for use as a live vaccine.

5. The strain of claim 3 or 4 for use as a vaccine to protect against a Salmonella infection.

6. The strain of any one of claims 1-5 for use in mammals, e.g. pigs, or birds, e.g. poultry such as chickens.

7. Use of a polypeptide comprising a Salmonella phoN polypeptide or/and any immunogenic portion thereof as a serological marker antigen, wherein the phoN polypeptide comprises the sequence SEQ ID NO:1 or a sequence which is at least 70% identical to SEQ ID NO: 1, and wherein the immunogenic portion has a length of preferably at least 10, more preferably at least 20 and even more preferably at least 30 amino acids.

8. The use of claim 7 for the differentiation of infected from vaccinated animal (DIVA) after vaccination with the strain of any one claims 1-6.

9. The use of claim 7 or 8, wherein the phoN antigen is prepared by recombinant expression of a phoN nucleic acid, particularly in a recombinant host cell such as E. coli.

10. The use of any one of claims 7-9, wherein the phoN antigen comprises the full-length phoN polypeptide of SEQ ID NO: 1.

11. A serological test system for detecting antibodies directed against a Salmonella phoN antigen, comprising at least one recombinant Salmonella phoN antigen or any immunogenic portion thereof and optionally further test components, wherein the phoN antigen comprises the sequence SEQ ID NO:1 or a sequence which is at least 70% identical to SEQ ID NO:1.

12. Use of a Salmonella phoN nucleic acid for the manufacture of a recombinant Salmonella phoN antigen or any immunogenic portion thereof, wherein the phoN antigen comprises the sequence SEQ ID NO:1 or a sequence which is at least 70% identical to SEQ ID NO:1.
